SEQUENCE LISTING (22 sequences)

SEQ ID NO: 1 - DNA, 20 bp, Artificial Sequence
  Synthetic primer
cccaccgtgttcttcgacat

SEQ ID NO: 2 - DNA, 20 bp, Artificial Sequence
  Synthetic primer
ccagtgctcagagcacgaaa

SEQ ID NO: 3 - DNA, 21 bp, Artificial Sequence
  Synthetic primer
ggagatcatagttccgtgagc

SEQ ID NO: 4 - DNA, 20 bp, Artificial Sequence
  Synthetic primer
gctgagatccatgccattgt

SEQ ID NO: 5 - DNA, 19 bp, Artificial Sequence
  Synthetic primer
ccgccacagatttacccgt

SEQ ID NO: 6 - DNA, 20 bp, Artificial Sequence
  Synthetic primer
agtctggtagcgcgtgtaac

SEQ ID NO: 7 - DNA, 20 bp, Artificial Sequence
  Synthetic primer
ccaggaccagaaagccagta

SEQ ID NO: 8 - DNA, 23 bp, Artificial Sequence
  Synthetic primer
gttaggtagcgattgaagtgaaa

SEQ ID NO: 9 - DNA, 20 bp, Artificial Sequence
  Synthetic primer
cctccgccaacactaacagt

SEQ ID NO: 10 - DNA, 20 bp, Artificial Sequence
  Synthetic primer
caaggtctgataccggctgt

SEQ ID NO: 11 - DNA, 20 bp, Artificial Sequence
  Synthetic primer
acctcctagcgtccaggttt

SEQ ID NO: 12 - DNA, 19 bp, Artificial Sequence
  Synthetic primer
cgaagctacaggacggaaa

SEQ ID NO: 13 - DNA, 19 bp, Artificial Sequence
  Synthetic primer
tcgctgaaggaggaggaga

SEQ ID NO: 14 - DNA, 21 bp, Artificial Sequence
  Synthetic primer
caaacacccacaaaggaaaac

SEQ ID NO: 15 - DNA, 21 bp, Artificial Sequence
  Synthetic primer
acctcggataacgaagcgaaa

SEQ ID NO: 16 - DNA, 20 bp, Artificial Sequence
  Synthetic primer
tccagcgtctggtgtttagt

SEQ ID NO: 17 - DNA, 19 bp, Artificial Sequence
  Synthetic primer
gaacctgtgcgagtggatg

SEQ ID NO: 18 - DNA, 20 bp, Artificial Sequence
  Synthetic primer
ggatggtgatgtagcgactg

SEQ ID NO: 19 - DNA, 19 bp, Artificial Sequence
  Synthetic primer
tctttgcttgggaaatccg

SEQ ID NO: 20 - DNA, 21 bp, Artificial Sequence
  Synthetic primer
ctgcccgttcaacatccttag

SEQ ID NO: 21 - DNA, 20 bp, Artificial Sequence
  Synthetic primer
gatgcacaactcggagatca

SEQ ID NO: 22 - DNA, 20 bp, Artificial Sequence
  Synthetic primer
gtccttcttgagcagcgtct

(19) United States
(12) Patent Application Publication
(10) Pub. No.: US 2023/0416675 A1
(43) Pub. Date: Dec. 28, 2023

(54) EFFICIENT AND RAPID SPECIFICATION OF SPINAL NEURONAL SUBTYPES FROM HUMAN PLURIPOTENT STEM CELLS

(71) Applicants: INSERM (INSTITUT NATIONAL DE LA SANTE ET DE LA RECHERCHE MEDICALE), Paris (FR); SORBONNE UNIVERSITE, Paris (FR); UNIVERSITÉ D'EVRY-VAL-D'ESSONNE, Evry (FR); CENTRE D'ETUDE DES CELLULES SOUCHES (CECS), Corbeil-Essonnes (FR); UNIVERSITÉ PARIS-SACLAY, Gif-sur-Yvette (FR)

(72) Inventors: Stéphane NEDELEC, Paris (FR); Cécile MARTINAT, Corbeil-Essones (FR); Vincent MOUILLEAU, Le Poirè-sur-Vie (FR); Célia VASLIN, Paris (FR)

(21) Appl. No.: 18/038,734

(22) PCT Filed: Nov. 25, 2020
(86) PCT No.: PCT/IB2020/000972

§ 371 Date: May 25, 2023

(51) Int. Cl. C12N 5/0793

(52) U.S. Cl. CPC C12N 5/0619; C12N 2506/45; C12N 2501/41; C12N 2501/19; C12N 2501/115; C12N 2501/16; C12N 2501/119; C12N 2501/415

(57) ABSTRACT

The present invention relates to the targeted engineering of specific cell populations. Motoneurons (MN) subtypes display differential vulnerabilities in diseases and in spinal injuries. Engineered MNs of specific rostro-caudal identity represent an important resource for cell therapy approaches. However, these strategies remain impeded by slow and inefficient targeted differentiations due to the imprecise control over cell fate specification in vitro. The inventors now used an embryoid body-based differentiation of hPSC and showed that the HOX clock expression can be controlled to generate subtypes of spinal MNs. Thus, the present invention relates to an in vitro or an ex vivo method for producing spinal neuronal subtypes comprising exposing axial progenitors to retinoic acid (RA), an agonist of Hedgehog signalling pathway, and optionally a FGFR agonist and/or an activator of the TGF pathway, wherein more and more caudal motor neurons identities are obtained by delayed exposure to RA and/or by exposure to RA in combination with the FGFR agonist and/or the activator of the TGF pathway.

The present invention concerns a method for the generation of human cell types having a defined rostro-caudal identity.

### BACKGROUND

The patterning of bilaterian body is orchestrated by the differential expression of HOX transcription factors along their rostro-caudal axis. The initiation of Hox gene expression patterns is closely linked to the rostro-caudal extension of the body axis.

As axial progenitors contribute to progressively more caudal mesodermal and neuroectodermal structures, the 3′ to 5′ sequence of HOX gene activation is translated into a collinear spatial pattern of expression with 3′ HOX genes being expressed anteriorly while 5′ HOX ones are late-activated and caudally-expressed (Deschamps and Duboule, 2017; Henrique et al., 2015). While extrinsic factors such as Retinoic Acid (RA), Wnt, or Fibroblast Growth Factors (FGFs) play a key role in controlling HOX patterns, cell intrinsic changes in chromatin states within the complexes correlate with the temporal sequence of their transcriptional induction (Bel-Vialar et al., 2002; Deschamps and Duboule, 2017; Liu et al., 2001; Mazzoni et al., 2013; Narendra et al., 2015; Neijts et al., 2017; Philippidou and Dasen, 2013). However, it remains unclear whether the progressive opening of the chromatin along the complexes serves as an internal timer initiated and terminated by extrinsic cues or whether sequences of secreted factors that activate progressively more caudal HOX genes define the tempo of the induction (Bel-Vialar et al., 2002; Del Corral and Storey, 2004; Deschamps and Duboule, 2017; Ebisuya and Briscoe, 2018; Lippmann et al., 2015; Mazzoni et al., 2013; Wymeersch et al., 2019). This might result in the current limited control over pluripotent stem cells (PSCs) differentiation into caudal cell types of distinct well-defined rostro-caudal identities (Diaz-Cuadros et al., 2020; Du et al., 2015; Duval et al., 2019; Faustino Martins et al., 2020; Frith et al., 2018; Gouti et al., 2014; Li et al., 2005; Lippmann et al., 2015; Matsuda et al., 2020; Maury et al., 2015; Ogura et al., 2018; Peljto et al., 2010; Verrier et al., 2018). Indeed, the two models imply different strategies to control in vitro PSC differentiation. The “intrinsic model” predicts that the efficient specification of posterior identities will rely on a precise synchronization between the differentiation timing and the internal HOX timer. Alternatively, the second mechanism predicts that exposing axial progenitors to the relevant extrinsic cues will entrain the HOX clock to generate progenies of defined rostro-caudal identities.

Spinal (sMN) motor neurons located in the spinal cord are one of two fundamental classes of motor neurons, the others being cranial motor neurons from the hindbrain. sMN relies on a precise HOX code to acquire appropriate rostro-caudal subtype identities. Indeed, the differential expression of HOX transcription factors along the vertebrate spinal cord is a major product of HOX gene regulation. In spinal motor neurons, this Hox code orchestrates the specification of subtype specific features controlling the formation of locomotor circuits (Dasen, 2017; Philippidou and Dasen, 2013). In particular, the Hox expression profile in motor neurons regulates their subtype specification, columnar and pool segmentation. and innervation targeting of muscle groups. However, whether the spinal HOX code and associated MN subtypes is conserved in human remains unknown, thus preventing faithful assessment of HOX regulation and its link with cell “rostro-caudal” identity during hPSC differentiation.

The targeted engineering of specific cell populations is a major avenue for developmental studies, disease modelling and regenerative medicine. However, these strategies remain impeded by slow and inefficient targeted differentiations due in large part to the imprecise control over cell fate specification in vitro.

In particular, MN subtypes display differential vulnerabilities in disease and in spinal injuries. Newly engineered motor neurons of specific rostro-caudal identity would thus represent a long-awaited resource for the modelling of these incurable diseases and more controlled source of cells for putative cell therapy approaches.

A previous application from the inventors (see WO2016012570) provided a method for obtaining enriched populations of sMNs and cMNs from hPSCs. It was notably shown that, by culturing neuralized hPSCs in the presence of high concentrations of activators of the Wnt signalling pathway, in combination with Retinoic Acid (RA) and SAG, it was possible to obtain a population comprising more than 80% of spinal motor neuron progenitors 10 days after the beginning of the culture, that could efficiently (60 to 70% of the cells) differentiate into spinal motor neurons in the presence of gamma-secretase inhibitors. However, while MN subtypes are differentially affected in diseases and no efficient methods existed to generate them from hPSCs, this application did not provide any clue regarding the specific rostro-caudal and subtype identity of sMNs nor any indication or method for obtaining such specific sMNs.

It has been also shown that retinoic acid (RA), wingless-type MMTV integration site protein family (Wnt), fibroblast growth factor (GDG) and growth differentiation factor (GDF) signalling intricately regulate HOX expression during posterior CNS development (Liu et al, 2001; Nordström et al., 2006). Currently the most efficient procedure to specify sMNs (Maury et al 2015, Du et al 2015 doi: 10.1038/ncomms7626) generate anterior cervical MNs. However, largely due to a poor knowledge of the combination of cues and the time of their action necessary to generate MN subtypes currently no efficient method to generate MN subtypes of defined rostro-caudal identity have been reported. In particular the generation of brachial, thoracic and lumbar MNs and in particular, among these MN subtypes, limb innervating MNs remain elusive. HOX mRNA profiles differ and are broader than the domain of their proteins. Thus, mRNA profiles, notably in motor neuron progenitors, do not reflect the Hox protein expression in spinal motor neurons. Consequently, the assessment of MN subtypes based on mRNA expression, for example by PCR analysis, thus lacks the required accuracy to identify MN subtypes (Dasen et al, Nature 2003, Lippmann et al., 2015). Poorly characterized antibodies in human have also impaired the proper characterization of MN subtypes. Furthermore, while attempts have been made, the resulting differentiation which relied on 2D adherent differentiation of hPSCS resulted in low percentage of MNs of mixed identities and often a contamination with non-neuronal cells demonstrating a poor control over the differentiation process. (Lippmann et al 2015, Patani et al, 2011). This limitation precludes systematic studies of MN subtypes in health and even more in disease in which selective MN populations are affected.

Accordingly, there is still an important need for improved an efficient procedure enabling obtaining enriched populations of sMNs with specific rostro-caudal phenotypes.

### SUMMARY OF THE INVENTION

The inventors have now used a embryoid body based differentiation of hPSC and provided evidence that the duration of the time window between Wnt and RA establishes the final positional identity of the progenies and that changes in the concentration, duration and combination of the caudalizing factors Fibloblast growth factor (FGF) and Growth differentiation factor (GDF) control the speed at which the temporal collinear activation of HOX genes occurs. The pace of the HOX clock is thus dynamically encoded by the parameters of exposure to extrinsic cues.

In a bioengineering perspective, this HOX clock extrinsic control provides a simple mean to engineer cell types of defined “rostro-caudal” identities from PSCs. It shortens temporal requirements for the generation of cells born at different time of development during axial elongation.

The method of the present invention therefore allows the synchronous engineering with an unprecedented efficiency and precision of human MN subtypes with defined rostro-caudal identities. In particular, the method allows to finely tune the rostro-caudal identity of sMNs.

The present invention therefore relates to an in vitro, or an ex vivo method for engineering human cell/neurons subpopulations of a defined rostro-caudal identity comprising culturing axial progenitors in a culture medium comprising retinoic acid (RA), an agonist of Hedgehog signalling pathway, and optionally a FGFR agonist and/or an the activator of the TGF/activin/nodal signalling pathway (including GDF11 or GDF8), wherein more and more caudal motor neurons identities are obtained by delayed addition, in the culture medium, of retinoic acid and/or by addition or retinoic acid in combination with the FGFR agonist and/or the activator of the TGF/activin/nodal signalling pathway.

More particularly, the present invention relates to an in vitro, or an ex vivo method comprising:
exposing pluripotent stem cell in a culture medium comprising an activator of the Wnt signalling pathway and advantageously inhibitors of the BMP and activin/TGB pathways to obtain axial progenitors,
exposing axial progenitors to retinoic acid (RA) and an agonist of the Hedgehog signalling pathway, optionally combined with a FGFR agonist and/or the activator of the TGF/activin/nodal signalling pathway in the culture medium to obtain spinal motor neuron progenitors, and
optionally exposing spinal motor neuron progenitors to a notch pathway inhibitor in the culture medium,
   wherein more and more caudal motor neurons identities are generated either by delaying exposure to retinoic acid with regard to exposition to the Wnt signalling pathway activator or by exposing axial progenitors to retinoic acid in combination with increased exposures to the FGFR agonist and/or the activator of the TGF/activin/nodal signalling pathway, wherein typically increased exposure involves an increased concentration in the culture medium and/or an increase duration in the culture medium.

Typically, the human pluripotent stem cells (hPSCs) are exposed to an inhibitor of the Bone Morphogenetic Protein (BMP) signalling pathway and an inhibitor of the Transforming Growth Factor (TGF)/activin/nodal signalling pathway, prior to, or in parallel with the activator of the Wnt signalling pathway.

Typically also, caudal thoracic and lumbar motor neurons are obtained by exposing axial progenitors to retinoic acid, the FGFR agonist and the activator of the TGF/activin/nodal signalling pathway.

Optionally, the FGFR agonist is added in the culture medium at a concentration of at least 15 ng/ml, notably ay least 50 ng/ml or at least 100 ng/ml for a period of time of at least 24 hours and the activator of the TGF/activin/nodal signalling pathway (such as GDF11 or GDF8), is added to the culture medium at a concentration of at least 20 ng/ml for a period of time of at least 24 hours.

In some embodiments, of the method of the invention:
the inhibitor of the Bone Morphogenetic Protein (BMP) signalling pathway, the inhibitor of the Transforming Growth Factor (TGF)/activin/nodal signalling pathway, and the activator of the Wnt signalling pathway are added in the culture medium starting from DO until about D3 or D4,
the agonist of the sonic Hedgehog signalling pathway can be added to the culture medium from about D3 to about D7 and until about D9, typically between D3 and D9, D4 and D9, D5 and D9, D6 and D9, D7 and D9, and
the notch pathway inhibitor is added between about D9 and D14 or more.

Typically, RA is added to the culture medium for a period of about 2 to 11 days, optionally between about D3 and D9, optionally at a concentration of at least 10 nM.

In some embodiments of the method:
brachial motor neurons are obtained by addition in the culture medium of RA alone between D4 and D9 or between D3 and D9 in combination with a FGFR agonist at a concentration of at least 100 ng/ml, or an activator of the TGF/activin/nodal signalling pathway at a concentration of at least 20 ng/ml, between D3 and D4;
anterior thoracic motor neurons are obtained by addition in the culture medium of RA alone starting from D5 or later until D9 (or later), or between D3 and D9 (or later) in combination with a FGFR agonist at a concentration of at least 60 ng or with an activator of the TGF/activin/nodal signalling pathway at a concentration of at least 20 ng/ml, starting from D3 and or 48 h or more (preferably 48 h).
caudal thoracic motor neurons are obtained by addition in the culture medium of RA between D3 and D9 and by a combination between D3 and D4 of a FGFR agonist at a concentration of at least 100 ng/ml and GDF11 at a concentration of at least 20 ng/ml;
lumbar motor neurons are obtained by addition in the culture medium of RA between D5 and D9 (or later) and by a combination a FGFR agonist at a concentration of at least 100 ng/ml and GDF11 at a concentration of at least 20 ng/ml added in the culture medium starting from D3 or D4 and until D5.

In some embodiments, the FGFR agonist is FGF2 or 8.

In some embodiments, the agonist of the sonic Hedgehog signalling pathway is the Smoothened Agonist (SAG), optionally the SAG is added at a concentration of at least 300 nM.

In some embodiments, the notch pathway inhibitor is DAFT, optionally at a concentration of at least 5 μM. The notch pathway inhibitor is typically added from around D9 to around D14 or later.

In some embodiments, the activator of the Wnt signalling pathway is selected from the compound Chir-99021 or the Wnt3a protein.

### DETAILED DESCRIPTION OF THE INVENTION

Unless defined otherwise, all technical and scientific terms used herein have the same meaning as commonly understood by one of ordinary skill in the art to which the invention pertains.

Unless specifically mentioned, the present invention encompasses any combinations of the various embodiments as herein described.

In describing the embodiments and claiming the invention, the following terminology will be used in accordance with the definitions set out below.

As used herein, “about” or “around” means within 10% of a stated concentration range or within 10% of a stated time frame.

As used herein, “effective amount” means an amount of an agent sufficient to evoke a specified cellular effect according to the present invention.

The previous study of Maury et al., identified spinal progenitors having putative characteristics of such axial progenitors as they were able to generate motor neurons, however neither the competence of these progenitors nor the rostro-caudal identity of their progeny had been established. Indeed, the expression pattern of the HOX transcription factor was so far not established and markers of pools and column of MNs in human spinal cord remained unknown. The inventors have now characterized the expression pattern of HOX transcription factors in human embryos. They have thus defined combinations of transcription factors usable as marker for distinct rostro-caudal positions in the spinal cord and identified molecular mechanism governing their specification.

The present invention thus provides a method for generating spinal motor neurons of defined rostro-caudal identities. It relates to the inventors' finding that under defined conditions, differentiation of hPSCs into axial progenitors by exposure to of an activator of Wnt signalling inhibitors, typically in combination with prior or parallel exposure to inhibitors of the BMP and TGF pathways, followed by timely application of a retinoic acid receptor agonist optionally combined with a FGFR agonist and an activator of the TGF/activin/nodal signalling pathway allows precise positional patterning of the resulting posterior neuroectoderm and neuroepithelium along the hindbrain-spinal axis, as reflected by HOX family gene expression.

The present invention relates to a method, in particular an in vitro, or an ex vivo method, for engineering human motor neuron subpopulations of a defined rostro-caudal identity comprising:
a) exposing human pluripotent stem cells (PSCs) to an activator of the Wnt signalling pathway, and advantageously to inhibitors of the BMP and activin/TGB pathways is/are added to obtain axial progenitors,
b) exposing the resultant axial progenitors to retinoic acid (RA) and an agonist of the sonic Hedgehog signalling pathway, optionally combined with a FGFR agonist and/or an activator of the TGF/activin/nodal signalling pathway, to obtain motor neurons progenitors, and
c) optionally exposing motor neuron progenitors to a notch pathway inhibitor in the to obtain post-mitotic human motor neurons;
   wherein more and more caudal motor neurons identities are generated either by delaying exposure to retinoic acid with regard to exposition to the Wnt signalling pathway activator or by exposing axial progenitors to retinoic acid in combination with increased exposures to the FGFR agonist and/or the activator of the TGF/activin/nodal signalling pathway, wherein typically increased exposure involve an increased concentration in the culture medium and/or an increase duration in the culture medium.

Various embodiments of this method which can be taken alone or in combination are illustrated in the detailed description below.

Identification of Molecular Markers According to the Present Invention

Typically, the method of the present invention allows to obtain enriched cell populations expressing the combination of molecular markers as herein disclosed, in particular as defined in table 2. Enriched cell populations refer to cell populations, ex vivo, that contain a higher proportion of a specified cell type or cells having a specified characteristic than are found in vivo (e.g., in a tissue).

Techniques to identify expression of molecular markers are well-known from the skilled person and include immunostaining, western blotting, and flow cytometry, using antibodies specific for said markers. mRNA expression levels in a population can be detected using any of a number of routine methods in the art including, but not limited to, qRT-PCR, RNA-blot, RNA sequencing, and RNAse protection.

Suitable quantitative methods for evaluating molecular markers of interest as described herein also include, well-known techniques such as qRT-PCR, RNA-sequencing, RNA-blot, RNAse protection, and the like for evaluating gene expression at the RNA level. Quantitative methods for evaluating expression of markers at the protein level in cell populations are also known in the art. For example, flow cytometry, is typically used to determine the fraction of cells in a given cell population expressing (or are not expressing) one or two protein markers of interest (e.g., Brachyury, and Sox2).

Pluripotent Stem Cells

As used herein, the term “pluripotent cells” refers to undifferentiated cells which can give rise to a variety of different cell lineages.

Preferably, the pluripotent cells are human pluripotent cells.

Preferably, the pluripotent cells are stem cells.

In the context of the present invention, stem cells encompass embryonic stem cells (ESCs); foetal stem cells including stem cells of the embryo proper and of extra-embryonic tissues such as amniotic fluid stem cells, as well as induced pluripotent stem cells. In particular, stem cells according to the invention encompass foetal stem cells and induced pluripotent stem cells.

The term “embryonic stem cells” refers to pluripotent stem cells derived from the epiblast tissue of the inner cell mass of a blastocyst or earlier morula stage embryos. Embryonic stem cells may also be defined by the expression of several transcription factors, such as Oct-4, Nanog and Sox2, and cell surface proteins such as the glycolipids SSEA3 and SSEA4 and the keratin sulphate antigens Tra-1-60 and Tra-1-81.

In a particular embodiment, the term “stem cells” in accordance with the invention does not comprise stem cells from human embryos. More particularly, the stem cells according to the invention are preferably not directly derived from a human embryo or did not necessitate the destruction of a human embryo. In particular, embryonic stem cells which have been derived from publicly available and previously established stem cell lines fall within the meaning of the term “stem cells” as used in the present invention, in particular embryonic stem cells which have been derived from publicly available and previously established stem cell lines which did not necessitate the destruction of a human embryo, as for example described in Chung et at. (2008) Cell Stem Cell 2:1 13-1 17).

In particular, human embryonic stem cells derived from one of the cell lines exemplified in Table 1 fall within the meaning of the term “stem cells” as used in the present invention.

TABLE 1
Human embryonic stem cell lines age available Country of origin

| | | Passage | Country of | |
|---|---|---|---|---|
| Line | Karyotype | available | origin | Origin |
| SAD1 | 46XY | 25 | Sweden | Cellartis AB |
| H9 | 46XX | 42 | USA | Wicell research |
| | | | | Institute |

The term “foetal stem cells” refers to stem cells derived either from the foetus proper or preferably from the extra-embryonic tissues emerging during gestation including umbilical cord blood, amniotic fluid, Wharton's jelly, the amniotic membrane and the placenta.

As used herein, the term “induced pluripotent stem cell” or “iPS cell” refers to a type of pluripotent stem cell artificially derived (e.g., induced by complete or partial reversal) from a differentiated cell (e.g. a non-pluripotent cell), typically an adult somatic cell such as an adult fibroblast.

In some embodiments, the stem cells used in the context of the invention are induced pluripotent stem cells. Suitable induced pluripotent stem cells (iPSCs) can typically be obtained from the European Bank for Pluripotent Stem Cells (EBISC), such as for example lines WTSIi002 (male, RRID: CVCL_AH30, alternative name HPSI0913i-eika_2) and. WTC-mEGFP-Safe harbor locus (AAVS1)-c16 produced by the Allen cell institute was obtained from Coriell (Cat #AICS-0036-006, male, RRID:CVCL_JM19).

In some embodiments, the pluripotent cells are obtained from an individual suffering from a neurodegenerative genetic disease, such as hereditary amyotrophic lateral sclerosis (ALS) or spinal muscular atrophy (SMA). In another embodiments, the pluripotent cells contain a genetic mutation for example responsible for a neurodegenerative genetic disease, such as hereditary amyotrophic lateral sclerosis (ALS) or spinal muscular atrophy (SMA). Advantageously, in these embodiments, the population of motor neuron progenitors and/or the population of motor neurons also contain said mutation and can therefore provide a good cellular model of the disease.

Advantageously, the human pluripotent stem cells used in the present invention are dissociated to single cells are let to re-aggregate in non-adhesive culture condition such as in microwell plates or in ultra-low adhesion plate, into free floating aggregate of PSC stem cells on day 0 (in the form of embryoid bodies also named aggregate of PSC). Such proceeding is described in the Material & Method section of the results. A protocol is also detailed in Maury et al (Maury Y, Côme J, Piskorowski R A, Salah-Mohellibi N, Chevaleyre V, Peschanski M, Martinat C, Nedelec S. Combinatorial analysis of developmental cues efficiently converts human pluripotent stem cells into multiple neuronal subtypes. Nat Biotechnol. 2015 January; 33(1):89-96).

Axial Progenitors

As used herein the term “axial progenitors” refers to progenitor cells derived from hPSCs that generate neural progenitors and the neurons of the spinal cord.

Axial progenitors are typically induced by adding a Wnt activator in the hPSCs culture medium allowing their conversion into axial progenitors competent to generate progenies expressing distinct HOX combinations that locate at different rostro-caudal positions in the spinal cord.

Axial progenitors can be notably characterized by the co-expression of SOX2, CDX2. In some embodiments, typically after 2 to 3 days exposure to the Wnt agonist, axial progenitors typically also coexpress TBXT (BRACHYURY or BRA). The SOX2+/CDX2+/BRA+ profil characterizes a population of axial progenitors that generate the spinal cord and somite in mouse embryos.

Typically, D2, D3 and D4 (i.e. from around 2 to at least 4 days after initiating exposure to the activator of the Wnt pathway), axial progenitors show a high enrichment expression of CDX1 and 2, TBXT (BRACHYURY), FGF17, RXRG, SP5/8, WNT5A/B, WNT8A, FGF8, GRSF1, CYSTM1, HES3 together with SOX2) associated with a loss of most pluripotency markers, such as Nanog, KIf2, Klf4, Prdm14, Fgf4, Oct4 (Also In Early NMP), Sox2 (Also In Caudal Epiblast And Neurectoderm).

Axial progenitors also typically do not express typical markers of node cells, mesodermal and allantois, such as Ccno, Nodal, Nog, Noto, Shh; Aldh1a2, Cited1, Meox), Msgn), Tbx3 and 6, Osr1, Pax2, Mesp1/2, Foxf1, Hand)-2; and Mixl1, Evx1 Gata6, Tbx2, Tbx4, Mixl1, Evx1 respectively (see notably FIG. 2B-C and Table S1 of the Results section as well as Cambray and Wilson, 2007; Edri et al., 2019; Gouti et al., 2014; Gouti et al., 2017; Henrique et al., 2015; Koch et al., 2017; Wymeersch et al., 2016; Wymeersch et al., 2019).

Motor Neurons and Progenitors

As used herein, the term “motor neuron” or “motoneuron” refers to an efferent neuron, which innervate a muscle cell or autonomic ganglia. Motor neurons include two fundamental classes: spinal motor neurons and cranial motor neurons.

As used herein, the term “spinal motor neuron” refers to a motor neuron located in the spinal cord, while “cranial motor neurons” refers to motor neurons located in the brainstem. Without any other specification the term “motor neuron” used in the present invention is also used by extension for “spinal motor neurons”. As well-known by the skilled person, spinal motor neurons can be characterized by the expression of a specific pattern of markers. Typically, spinal motor neurons can be characterized by being notably ISL1/2+(ISL LIM Homeobox 1 or 2 positive), and/or HB9+(Homeobox protein 9 positive). Additional markers that can be expressed by spinal motor neurons include Lhx3 (LIM homeobox 3), FOXP1 (forkhead box P1), Lhx1 (LIM homeobox 1), CHAT (Choline acetyltransferase) and VACHT (vesicular acetylcholine transporter). Identification of spinal motor neurons can thus be carried out by any technique well-known from the skilled person involving the detection of the above markers, such as immunostaining using for example specific anti-ISL1/2 and/or anti-HB9 antibodies.

Spinal motor neurons, more particularly, functional spinal motor neurons can also be characterized by using electrophysiology techniques, such as whole-cell patch clamp recording. Typically, whole-cell patch clamp assays can be performed as follows: patch pipettes (3-4 MA) containing 135 mM KMethylSulfate, 5 mM KCl, 0.1 mM EGTA-Na, 10 mM HEPES, 2 mM NaCl, 5 mM ATP, 0.4 mM GTP, 10 mM phosphocreatine (pH 7.2; 280-290 mOsm) and an extracellular solution containing (in mM) 125 mM NaCl, 2.5 mM KCl, 10 mM glucose, 26 mM NaHC0₃, 1.25 mM NaH₂P0₄, 2 mM Na Pyruvate, 2 mM CaCI₂ and 1 mM MgCI₂ and saturated with 95% O₂ and 5% CO₂ are used, at a pH of 7.3. Membrane potentials are corrected for liquid junction potential, which can be typically measured to be −3 mV. Series resistance (typically less than 10 MΩ) are monitored and compensated throughout each experiment with the amplifier circuitry. 0.5 μM tetrodotoxin (TTX) is bath-applied following dilution into the external solution from concentrated stock solutions. To maintain constant osmolarity and pH, 30 mM TEACI (tetraethylammonium chloride) can be added to a modified external solution (95 mM NaCl instead of 125 mM). Glutamate can be added with equal concentration NaOH to maintain neutral pH. Leak current is subtracted from current families using a positive/negative (P/N) protocol and each set of currents is for example averaged four times. All data can be acquired with Axograph X software and analyzed for example with Igor pro. Functional immature spinal motor neurons can thus be characterized by firing small immature spikes in response to, for example a 600 pA current injection with shape and magnitude typically from 3 to 10 mV. Functional mature spinal motor neurons can be characterized by firing trains of action potentials with shape and magnitude typically from 60 to 80 mV, (see international application PCT/EP2015/066944). Functional mature spinal motor neurons may also be characterized by detecting choline acetyl transferase (CHAT) expression, using conventional immunostaining and Western Blot techniques.

The term “motor neuron progenitor” or “motoneuron progenitor” refers to a cell population that can give rise to one of the two main groups of motoneurons (i.e., spinal motor neurons and cranial motor neurons). The term “spinal motor neuron progenitor” typically refers to Olig2 positive cells. As well-known from the skilled person, spinal motor neurons progenitors can be characterized by the expression of a specific pattern of markers. Typically, generated spinal motor neuron progenitors can be characterized by being OLIG2+(oligodendrocyte lineage transcription factor 2 positive), NKX6.1+(NK6 homeobox 1 positive), SOX1+ and SOX2+.

Identification of spinal motor neuron progenitors can thus be carried out by any technique well-known from the skilled person involving the detection of the above markers, such as typically immunostaining using specific anti-OLIG2 and/or anti-NKX6.1 antibodies, SOX1 and 2. Subtypes of MN progenitors along the rostro-caudal axis can be characterized by the expression of HOX mRNAs by RT-PCR (and immunostaining for HOXC9)(see below and notably in the result section).

Human Spinal Motor Neuron (hsMNs) Rostro-Caudal Subpopulations

The inventors showed in the results included herein that HOX transcription factors are regionally expressed along the rostro-caudal axis of the human spinal cord. Within these different HOX domains, distinct MN subtypes, identifiable by combination of transcription factors, are generated at stereotyped positions.

It has been shown that (post-mitotic) human MNs express ISL1 and/or HB9 all along the spinal cord. It has been further demonstrated that within MNs, specific HOX expression displayed rostro-caudal patterns: cervical MNs expressed HOXA/C5, while brachial MNs expressed HOXC6, thoracic MNs HOXC9 and lumbar MNs HOXC10. Caudal brachial MNs co-expressed HOXC6 and HOXC8 and anterior thoracic ones HOXC8 and HOXC9. HOXD9 labeled caudal thoracic MNs as well as anterior lumbar MNs together with HOXC10.

The inventors also showed that MNs expressing high level of FOXP1 (FOXP1^{high}) can be observed at brachial, anterior thoracic (HOXC6 and HOXC8/HOXC9) and lumbar (HOXC10) levels. They formed a lateral motor column (LMC) where limb innervating MNs are located. Within this FOXP1^{high} LMC, SCIP/HOXC8 MNs were observed in the caudal brachial spinal cord. In contrast, SCIP/HOXC8/HOXC9 MNs were located in the anterior thoracic region. Their location and their transcriptional code identify them as putative hand-controlling MNs

Thus, in some embodiments of the method of the invention, the subpopulation of spinal motor neurons (including spinal motor neurons progenitors) identified in table 2 below can be obtained.

TABLE 2
HOX expression profiles in MNs subtypes

| MNs subtypes | Molecular Characterization |
|---|---|
| brachial MNs | HOXC6⁺, HOXC8⁺/negative for HOXC9 |
| including | HOXC6⁺, HOXC8⁺, FOXP1^{high}/HOXC9⁻ |
| limb innervating | |
| MNs | |
| Anterior thoracic MNs | HOXC8+, HOXC9+/negative for HOXC6 |
| including | |
| limb innervating | HOXC8/HOXC9 + FOXP1 high/negative for |
| MNs | HOXC6 |
| Caudal thoracic MNs | HOXC9+, HOXD9+/negative HOXC6, HOXC8 |
| lumbar MNs | HOXC10/negative HOXC6, HOXC8, HOXC9 |
| including | |
| arm innervating | HOXC10 + FOXP1 high/negative HOXC6, |
| MNs | HOXC8, HOXC9 |

In some embodiments, at least 50% of the cells in an isolated population of human spinal motor neurons, obtained as per the present as herein disclosed, express one of the above-described Hox transcription factor profiles, e.g., 55%, 60%, 65%, 70%, 75%, 80%, 85%, 90%, 95%, 99%, or another percent of cells in the said isolated cell population exhibit the desired Hox transcription factor profile.

Activators of the Wnt Signalling Pathway and Obtention of Axial Progenitor

In the context of the invention, the term “activator of the Wnt signalling pathway” refers to any compound, natural or synthetic, which results in an increased activation of the Wnt signalling pathway, which is the series of molecular signals generated as a consequence of any member of the Wnt family binding to a cell surface receptor. Typically, an activator of the Wnt signalling pathway provokes an accumulation of β-catenin in the cytoplasm and its eventual translocation into the nucleus, as described in Bienz (2005) Curr. Biol. 15:R64-67. Techniques to determine whether a given compound is an activator of the Wnt signalling pathway are well-known from the skilled person. Typically, a compound is deemed to be an activator of the Wnt signalling pathway if, after culturing cells in the presence of said compound, the level of nuclear β-catenin is increased compared to cells cultured in the absence of said compound. Levels of nuclear β-catenin can be measured by Western blot using antibodies specific for β-catenin.

The activator of the Wnt signalling pathway may be a Wnt agonist or a molecule which activates any downstream step of the Wnt signalling pathway. The activator of the Wnt signalling may be a natural or a synthetic compound. When the activator of the Wnt signalling pathway is a protein, it may be a purified protein or a recombinant protein or a synthetic protein.

Examples of activators of the Wnt signalling pathway include but are not limited to the group consisting of the compound CHIR-99021 (6-[[2-[[4-(2,4-dichlorophenyl)-5-(5-methyl-1H-imidazol-2-yl)-2 pyrimidinyl]amino]ethyl]amino]-3-pyridinecarbonitrile, as described in Bennett et al. (2002) J. Biol. Chem. 277:30998-3104), the WNT3A protein, the compound IQ-1 (2-(4-Acetylphenylazo)-2-(3,3-dimethyl-3,4-dihydro-2H-isoquinolin-1-ylidene)-acetamide, as described in Miyabayashi et al. (2007) Proc. Natl. Acad. Sci. USA 104:5668-5673), the compound SB-216763 (3-(2,4-Dichlorophenyl)-4-(1-methyl-1H-indol-3-yl)-1H-pyrrole-2,5-dione as described in Coghlan et al. (2000) Chem. Biol. 7:793-803) and the compound BIO (6-bromoindirubin-3′-oxime, as described in Sato et al. (2004) Nat. Med. 10:55-63).

Preferably, the activator of the Wnt signalling pathway is selected from the group consisting of the compound CHIR-99021 and the WNT3A protein. Most preferably, the activator of the Wnt signalling pathway is the compound CHIR-99021.

Typically, the compound CHIR-99021 is added in the culture medium in a concentration ranging from 0.5 to 5 μM, preferably ranging from 1 to 4 μM, even more preferably at about 3 μM or at about 1 μM.

Typically, the Wnt activator is added in a culture of hPSCs, in particular in a culture of human embryoid bodies (as identified above, but see also Maury et al., as well as International application PCT/EP2015/066944 incorporated herein by reference) for a period of time sufficient to detect the expression of markers of axial progenitors as above mentioned and in particular the expression of CDX2, in particular for a period of time sufficient to obtain embryoid bodies having a SOX2+/CDX2+/BRA+ profile.

Embryoid bodies can be obtained using techniques well-known from the skilled person. Typically, pluripotent cells can be submitted to centrifugation, typically in V shape 384, for example at 1200 g for 5 min, to force cell aggregation and form embryoid bodies.

Thus, in some embodiments, the Wnt activator is typically applied in the culture medium for a period a time ranging from 2 to 4 days (optionally as detailed below together with inhibitors of of BMP and TGFβ pathways), notably ranging from 3 to 4 days, and typically for 4 days. According to the method of the present invention, the day of addition of the Wnt activator to the cultured embryoid bodies can be considered as Day 0 (D0).

Exposure of Embryoid Bodies to Inhibitors of the Bone Morphogenetic Protein (BMP) Signalling Pathway and of the Transforming Growth Factor (TGF)/Activin/Nodal Signalling Pathway

In some embodiments, hPSCs, and in particular human embryoid bodies, as defined in the section “Pluripotent cells”, are exposed to both (i) an inhibitor of the Bone Morphogenetic Protein (BMP) signalling pathway and (ii) an inhibitor of the Transforming Growth Factor (TGF)/activin/nodal signalling pathway (also shortly named TGF pathway in the present application).

As used herein, the term “inhibitor of the BMP signalling pathway” refers to any compound, natural or synthetic, which results in a decreased activation of the BMP signalling pathway, which is the series of molecular signals generated as a consequence of any member of the BMP (bone morphogenetic protein) family binding to a cell surface receptor. Typically, an inhibitor of the BMP signalling pathway provokes a decrease in the levels of phosphorylation of the proteins Smad 1, 5 and 8, as described in Gazzero and Minetti (2007) Curr. Opin. Pharmacol. 7:325-333. Techniques to determine whether a given compound is an inhibitor of the BMP signalling pathway are well-known from the skilled person. Typically, a compound is deemed to be an inhibitor of the BMP signalling pathway if, after culturing cells in the presence of said compound, the level of phosphorylated Smad 1, 5 or 8 is decreased compared to cells cultured in the absence of said compound. Levels of phosphorylated Smad proteins can be measured by Western blot using antibodies specific for the phosphorylated form of said Smad protein.

The inhibitor of the BMP signalling pathway may be a BMP antagonist or a molecule which inhibits any downstream step of the BMP signalling pathway. The inhibitor of the BMP signalling may be a natural or a synthetic compound. When the inhibitor of the BMP signaling pathway is a protein, it may be a purified protein or a recombinant protein or a synthetic protein.

The inhibitor of the BMP signalling pathway may be selected from the group consisting of noggin, chordin, follistatin, inhibitory Smad 6 (1-Smad 6), inhibitory Smad 7 (I-Smad 7), dorsomorphin (6-[4-(2-Piperidin-1-ylethoxy)phenyl]-3-pyridin-4-ylpyrazolo[1,5-a]pyrimidine; described in Yu et al. (2008) Nat Chem Biol. 4:33-41), the compound DMH1 (4-(6-(4-isopropoxyphenyl)pyrazolo[1,5-a]pyrimidin-3-yl)quinoline; described in Hao et al. (2010) ACS Chem Biol. 5:245-253), the compound DMH2 (4-(2-(4-(3-(quinolin-4-yl)pyrazolo[1,5-a]pyrimidin-6-yl)phenoxy)ethyl)morpholine; described in Hao et al. (2010) ACS Chem Biol. 5:245-253), the compound DMH3 (A/,/V-dimethyl-3-(4-(3-(quinolin-4-yl)pyrazolo[1,5-a]pyrimidin-6-yl)phenoxy)propan-1-amine; described in Hao et al. (2010) ACS Chem Biol. 5:245-253), the compound DMH4 (4-(2-(4-(3-phenylpyrazolo[1,5-a]pyrimidin-6-yl)phenoxy)ethyl)morpholine; described in Hao et al. (2010) ACS Chem Biol. 5:245-253), the compound LDN193189 (or DM-3189, 4-[6-(4-Piperazin-1-ylphenyl)pyrazolo[1,5-a]pyrimidin-3-yl]quinoline; described in Cuny et al. (2008) Bioorg. Med. Chem. Lett. 18:4388-4392), and the compound K02288 (3-[6-amino-5-(3,4,5-trimethoxy-phenyl)-pyridin-3-yl]-phenol; described in Sanvitale et al. (2013) PLoS ONE 8:62721), Preferably, the inhibitor of the BMP signalling pathway is the compound LDN193189.

Typically, the compound LDN193189 is added the culture medium comprising hPSCs in a concentration ranging from 0.05 to 1 μM, preferably from 0.1 to 0.5 μM, from 0.15 to 0.25 μM, even more preferably at about 0.2 μM.

As used herein, the term “inhibitor of the TGF/activin/nodal signalling pathway” refers to any compound, natural or synthetic, which results in a decreased activation of the TGF/activin/nodal signalling pathway, which is the series of molecular signals generated as a consequence of any member of the TGF/activin/nodal family binding to a cell surface receptor. Typically, an inhibitor of the TGF/activin/nodal signalling pathway provokes a decrease in the levels of phosphorylation of the protein Smad 2, as described in Shi and Massague (2003) Cell 113:685-700. Techniques to determine whether a given compound is an inhibitor of the TGF/activin/nodal signalling pathway are well-known from the skilled person. Typically, a compound is deemed to be an inhibitor of the TGF/activin/nodal signalling pathway if, after culturing cells in the presence of said compound, the level of phosphorylated Smad 2 is decreased compared to cells cultured in the absence of said compound. Levels of phosphorylated Smad proteins can be measured by Western blot using antibodies specific for the phosphorylated form of said Smad proteins.

The inhibitor of the TGF/activin/nodal signalling pathway may be a TGF/activin/nodal antagonist or a molecule which inhibits any downstream step of the TGF/activin/nodal signalling pathway. The inhibitor of the TGF/activin/nodal signalling may be a natural or a synthetic compound. When the inhibitor of the TGF/activin/nodal signalling pathway is a protein, it may be a purified protein or a recombinant protein or a synthetic protein.

The inhibitor of the TGF/activin/nodal signalling pathway may be selected from the group consisting of the compound SB431542 (4-(5-Benzol[1,3]dioxol-5-yl-4-pyrlidn-2-yl-1H-imidazol-2-yl)-benzamide hydrate), the Lefty-A protein and Cerberus.

Preferably, the inhibitor of the TGF/activin/nodal signalling pathway is the compound SB431542.

Typically, the compound SB431542 is added to the culture medium comprising hPSCs in a concentration ranging from 10 to 75 μM, preferably ranging from 20 to 50 μM, even more preferably at about 40 μM.

In a particularly preferred embodiment, the inhibitor of the BMP signalling pathway is the compound LDN193189 and the inhibitor of the TGF/activin/nodal signalling pathway is the compound SB431542. Typically, the compound LDN193189 is present in the culture medium at a concentration ranging from 0.15 to 0.25 μM, for example of about 0.2 μM and the compound SB431542 is present in the culture medium at a concentration ranging from 20 to 50 μM, for example of about 40 μM.

The inhibitor of the BMP signalling pathway and the inhibitor of the TGF/activin/nodal signalling pathway are applied to the culture medium for a period of time of about 2 to 4 days, notably 3 to 4 days and typically around 4 days.

Advantageously also, the PSCs and axial progenitors, which are submitted to neuralization following retinoic acid are in the form of embryoid bodies as above described. Embryoid bodies can be obtained from hPSCs using techniques well-known from the skilled person. Typically, pluripotent stem cells can be submitted to centrifugation, notably in V shape 384, for example at 1200 g for 5 min, to force cell aggregation and form embryoid bodies.

Advantageously the inhibitor of the BMP signalling pathway and the inhibitor of the TGF/activin/nodal signalling pathway are applied to the culture medium comprising PSCs (typically in the form of embryoid bodies together with the Wnt activator. Thus, the culture medium typically comprises the inhibitor of BMP signalling pathway, the inhibitor of the TGF/activin/nodal signalling pathway, and the activator of the Wnt signalling pathway as defined above. Thus, in some embodiments, hPSCs (notably in the form of human embryoid bodies or floating aggregates) are cultured in a culture medium wherein an inhibitor of BMP signalling pathway and an inhibitor of the TGF/activin/nodal signalling pathway are added, together with a Wnt activator or preceded by the addition of the Wnt activator.

In preferred embodiments, PSCs typically in the form of embryoid bodies (see above) are cultured in a culture medium wherein the Wnt activator is added to the culture medium together with the inhibitor of BMP signalling pathway and the inhibitor of the TGF/activin/nodal signalling pathway. In such embodiments, the inhibitor of the BMP signalling pathway and the inhibitor of the TGF/activin/nodal signalling pathway (as detailed above) can be added to the culture medium for a period of time ranging from 3 to 4 days (i.e. from DO to D3 or from DO to D4), in particular the inhibitor of the TGF/activin/nodal signalling pathway can be added from DO until D3, while the inhibitor of the BMP signalling pathway can be added (maintained) in the culture medium until D3 or D4, and the Wnt activator can be added to the culture medium for a period of time ranging from 2 to 4 day, notably from 3 to 4 days, (i.e., from DO to D2, from DO to day 3, or from DO to D4, preferably from DO to D3 or N4). Typically the inhibitor of the TGF/activin/nodal signalling pathway is not present any more in the culture medium after D3, when RA and/or the FGFR agonist and/or the activator of TGF/activin/nodal signalling pathway is present in the culture medium.

Retinoic Acid and Agonist of the Hedgehog Signalling Pathway:

As used herein, the term “retinoic acid” (RA) refers to an active form (synthetic or natural) of vitamin A, which is known to be capable of inducing neural cell differentiation. Examples of retinoic acid forms which can be used in accordance with the invention include, but are not limited to, retinoic acid, retinol, retinal, 11-cis-retinal, all-trans retinoic acid (ATRA), 13-cis retinoic acid and 9-cis-retinoic acid.

Typically, retinoic acid is added to the culture medium at a concentration ranging from 5 to 1000 nM, preferably ranging from 10 to 500 nM, or ranging from 100 nM to 500 nM. Typically, RA is added to the culture medium at a concentration of at least 100 nM.

A number of retinoic acid receptor agonists can also be used in the method of the present invention; An exemplary class of suitable retinoic acid receptor agonists are the retinoids and retinoid analogs, which include without limitation All-Trans Retinoic Acid (ATRA), Retinol Acetate, EC23 (4-[2-(5,6,7,8-Tetrahydro-5,5,8,8-te-tramethyl-2-naphthalenyl)ethynyl)-benzoic acid; CAS No: 104561-41-3), BMS453 (4-[(lE)-2-(5,6-Dihydro-5,5-dimethyl-8-phenyl-2-naphthalenyl)ethenyl]-benzoic acid; CAS No: 166977-43-10), Fenretinide (N-(4-Hydroxyphenyl)retinamide; CAS No: 65646-68-6), AM580 (4-[(5,6,7,8-Tetrahydro-5,5,8,8-tetramethyl-2-naphthalenyl)carboxamido]benzoic acid; CAS No: 102121-60-8), Tazarotene (6-[2-(3,4-Dihydro-4,4-dimethyl-2H-1-benzothiopyran-6-yl)ethynyl]-3-pyridinecarboxylic acid ethyl ester, CAS No: 118292-40-3), and TTNPB (4-[(E)-2-(5,6,7,8-Tetrahydro-5,5,8,8-tetramethyl-2-naphthalenyl)-1-propenyl]benzoic acid; CAS No: 71441-28-6). Other exemplary retinoic receptor agonists that could be used include AC261066 (4-[4-(2-Butoxyethoxy-)-5-methyl-2-thiazolyl]-2-fluorobenzoic acid; CAS No: 870773-76-5), AC55649 (4′-Octyl-[1,1′-biphenyl]-4-carboxylic acid; CAS No: 59662-49-6), Adapalene (6-(4-Methoxy-3-tricyclo[3.3.1.13,7]dec-1-ylphenyl)-2-naphthalenecarboxylic acid; CAS No: 106685-40-9), AM80 (4-[[(5,6,7,8-Tetrahydro-5,5,8,8-tetramethyl-2-naphthalenyl)amino]carbonyl]benzoic acid; CAS No: 94497-51-5), BMS753 (4-[[(2,3-Dihydro-1,1,3,3-tetramethyl-2-oxo-1H-inden-5-yl)carbonyl]amino]benzoic acid; CAS No: 215307-86-1), BMS961 (3-Fluoro-4-[[2-hydroxy-2-(5,5,8,8-tetramethyl-5,6,7,8,-tetrahydro-2-naphthalenyl)acetyl]amino]-benzoic acid; CAS No: 185629-22-5), CD1530 (4-(6-Hydroxy-7-tricyclo[3.3.1.13,7]dec-1-yl-2-naphthalenyl)benzoic acid; CAS No: 107430-66-0), CD2314 (5-(5,6,7,8-Tetrahydro-5,5,8,8-tetramethyl-2-anthracenyl)-3-thiophenecarboxylic acid; CAS No: 170355-37-0), CD437 (6-(4-Hydroxy-3-tricyclo[3.3.1.13,7]dec-1-ylphenyl)-2-naphthalenecarboxylic acid; CAS No: 125316-60-1), and Ch55 (4-[(lE)-3-[3,5-bis(1,1-Dimethylethyl)phenyl]-3-oxo-1-propenyl]benzoic acid; CAS No: 110368-33-7).

In the context of the invention, the term “agonist of the Hedgehog signalling pathway” refers to any compound, natural or synthetic, which results in an increased activation of the Hedgehog signalling pathway, which is the series of molecular signals generated as a consequence of any member of the Hedgehog family binding to a cell surface receptor. Typically, an agonist of the Hedgehog signalling pathway provokes activation of Hedgehog signalling pathway.

The agonist of the Hedgehog signalling pathway may be a Hedgehog agonist or a molecule which activates any downstream step of the Hedgehog signalling pathway. The agonist of the Hedgehog signalling may be a natural or a synthetic compound. When the agonist of the Hedgehog signalling pathway is a protein, it may be a purified protein or a recombinant protein or a synthetic protein.

Examples of agonists of the Hedgehog signalling pathway include but are not limited to the group consisting of purmophamine (9-cyclohexyl-N-[4-(morpholinyl)phenyl]-2-(1-naphthalenyloxy)-9H-purin-6-amine; as described in Sinha and Chen (2006) Nat. Chem. Biol. 2:29-30), SHH (sonic hedgehog), SHH C241 1 (as described in Taylor et al. (2001) Biochemistry 40:4359-4371), the smoothened agonist SAG (N-Methyl-N′-(3-pyridinylbenzyl)-N′-(3-chlorobenzo[b] thiophene-2-carbonyl)-1,4-diaminocyclohexane; as described in Chen et al. (2002) Proc. Natl. Acad. Sci. USA 99:14071-14076), and the compound Hh-Ag1 0.5 (3-chloro-4,7-difluoro-N-(4-methoxy-3-(pyridin-4-yl) benzyl)-N-(4-(methylamino) cyclohexyl) benzo[b] thiophene-2-carboxamide; as described in Frank-Kamenetsky et al. (2002) J. Biol. 1:10.2-10.19).

Preferably, the agonist of the Hedgehog signalling pathway is selected from SHH (Sonic Hedgehod) or SAG (Smoothened agonist). Most preferably, the agonist of the Hedgehog signalling pathway is SAG.

In some advantageous embodiments, the agonist of the Hedgehog signalling pathway, notably SAG, is added to the culture medium at a concentration of at least 300 nM, notably between 100 nM and 1 μM, or 100 to 1 μM, preferably ranging from 300 to 750 nM, even more preferably at about 500 nM.

Typically, the agonist of the Hedgehog signalling pathway is added to the culture medium for a period of time sufficient to induce the expression of markers of motor neuron progenitors, such as OLIG2n and NKX6.1. Techniques to detect the expression of markers of motor neuron progenitors are well-known from the skilled person and include immunostaining, western blotting, and RT-PCR, using antibodies specific for said markers, such as anti-OLIG2 and/or anti-NKX6.1 antibodies, or primers to detect NKX6. 1. Typically, this period of time may last for at least 2 days, more preferably for about 2 to 6 days, notably 2 to 5 days, typically for about 5 days.

In some embodiments of the method of the invention, the agonist of the Hedgehog signalling pathway is typically added to the culture medium at least 2 to 7 days after the beginning of the culture of hPSCs with the culture medium comprising the activator of the Wnt signalling pathway. Advantageously, the agonist of the Hedgehog signalling pathway is added to the culture medium between day D3 and D9, between day 4 (D4) and day 9 (D9), between D5 and D9, between D6 and D9 or between D7 and D9, preferably between D3 and D9.

Markers of neural plate identity are well-known from the skilled person and include typically SOX1, PAX6. Such markers are efficiently induced post Retinoic acid exposure.

Exposure of axial progenitors to the combination of RA and the agonist of the Hedgehog signalling pathway allows to generate spinal motor neuron progenitors as previously defined.

In some embodiment, post-mitotic spinal motor neurons can be rapidly obtained by exposing the spinal motor neuron progenitors to a differentiation culture medium comprising inhibitors of the Notch signalling pathway, in particular gamma secretase inhibitors.

As used herein, the term “inhibitor of the Notch signalling pathway” refers to any compound, natural or synthetic, which results, directly or indirectly, in a decreased activation of the Notch signalling pathway, which is the series of molecular signals generated as a consequence of the binding of a ligand to any member of the Notch family. Preferably, the inhibitor of the Notch signalling pathway is an inhibitor of gamma secretase. As well-known from the skilled person, gamma secretase inhibitors indirectly inhibit Notch pathway. Techniques to determine whether a given compound is an inhibitor of gamma secretase are well-known from the skilled person and are for example described in Yang et al. (2008) Molecular Brain 1:15 or Wang et al. (2009) Molecules 14:3589-2599.

The inhibitor of the Notch signalling, in particular the gamma-secretase inhibitor, may be a natural or a synthetic compound. When the inhibitor of the Notch signalling pathway is a protein, it may be a purified protein or a recombinant protein or a synthetic protein.

The inhibitor of the Notch signalling pathway may be selected from the group consisting of gamma-secretase inhibitors, in particular DAPT (N—[N-(3,5-Difluorophenacetyl)-L-alanyl]-S-phenylglycine t-butyl ester, as described in Borghese et al. (2010) Stem Cells 28:955-964), the compound W (3,5-Bis(4-nitrophenoxy)benzoic acid; as described in Okochi et al. (2006) J. Biol. Chem. 281:7890-7898), the compound L-685,458 ((5S)-(tert-Butoxycarbonylamino)-6-phenyl-(4/^{:}?)-hydroxy-(2/^{:}?)-benzylhexanoyl)-L-leucy-L-phenylalaninamide; as described in Shearman et al. (2000) Biochemistry 39 8698).

Preferably, the inhibitor of the Notch signalling pathway is the compound DAPT.

Typically, DAPT is present in differentiation culture medium in a concentration ranging from 1 to 25 μM, more preferably ranging from 5 to 20 μM, notably at about 10 μM.

The differentiation culture medium may also comprise other compounds known to contribute to the induction of the differentiation of motor neuron progenitors into motor neurons. Such compounds are well-known from the skilled person. Preferably, the culture medium C₃ further comprises BDNF (brain-derived neurotrophic factor) and GDNF (glial cell derived neurotrophic factor). Preferably, BDNF and GDNF are present in the differentiation culture medium each at a concentration of 5 to 100 ng/ml, preferably 10 to 50 ng/ml, more preferably each at about 10 ng/ml.

In the context of the invention, the period of time for exposing the spinal motor neurons progenitors to the differentiation culture medium is a period of time sufficient to induce the expression of makers of motor neurons, in particular of HB9, ISL1/2, HOXA5, HOXA4, and optionally Lhx3 or FOXP1. Techniques to detect the expression of markers of motor neuron are well-known from the skilled person and include immunostaining, flow cytometry, RT-PCR or in situ hybridization. Typically, the period of time may last for at least 5 days, preferably for 5 to 7 days, more preferably for about 5 days. Typically the inhibitor of the Notch signalling pathway is added 9 days after the beginning of the culture of hPSCs in the culture medium comprising the activator of the Wnt signalling pathway and in maintained in the culture medium for at least 5 to 7 days, notably for 5 days.

In some embodiments, the period of time of culture in the presence of retinoic acid and an agonist of the Hedgehog signalling pathway and the period of time of culture in the presence of an inhibitor of the Notch signalling pathway can overlap partially. Accordingly, during the periods when said periods of time overlap, the culture differentiation medium also comprises retinoic acid and an agonist of the Hedgehog signalling pathway.

Obtention of Defined Rostro-Caudal Phenotypes of sMNs

The inventors have demonstrated that motor neurons with more and more caudal identities can be generated by exposing axial progenitors to an agonist of the Hedgehog signalling pathway and to retinoic acid:
1. either by delaying RA exposure, or
2. by further exposing axial progenitors to a activator of the FGF pathway (FGFR agonists) and/or an activator of the TGF/activin/nodal signalling pathway (also shortly named herein TGF pathway).

A number of different fibroblast growth factor FGF isoforms can be used according to the present invention as FGFR agonists. Non-limiting examples of suitable FGFR agonists include FGF1, FGF2, FGF3n FGF8a, FGF8b, FGF10, FGF22, FGF8f, FGF17, and FGF18, typically the FGFR agonist is FGF2 or FGF8 (including FGF8a and FGF8b), notably FGF2.

The TGF activin/nodal signalling pathway notably involves activation Smad2 and Smad3 in response to activin, TGF-β and Nodal ligands interaction with type I receptors ALK4, ALK5 and ALK7. Thus, the present invention includes type I receptors ALK4, ALK5 and ALK7 agonists such as TGFβ family members (including TGFβs, activins and GDFs) and Nodal ligands. Non-limitative examples of such activators of the TGF pathway include GDF11 or GDF8.

Thus, retinoic acid is typically added to the culture medium at least 3 to 11 days and advantageously at least until day 9 (D9) after the beginning of the culture of hPSCs with the culture medium comprising the activator of the Wnt signalling pathway.

According to the present invention:

Brachial motor neurons, including FOXP1 high innervating motor neurons, can be obtained:
1. by addition in the culture medium of RA around D3 and until around D9 after the beginning of the culture of hPSCs in the culture medium comprising the activator of the Wnt signalling pathway, or
2. by addition in the culture medium of RA around D3 and until around D9 after the beginning of the culture of hPSCs in the culture medium comprising the activator of the Wnt signalling pathway in combination with the addition of a FGFR agonist, or an activator of the TGF/activin/nodal signalling pathway (typically GDF11 or GDF8) between D3 and D4, optionally wherein the FGFR agonist is added to the culture medium at a concentration of at least 100 ng/ml, notably between 100 and 250 ng/ml, between 100 and 200 ng/ml, preferably between 100 and 150 ng/ml, notably at about 120 ng/ml, or wherein the activator of the TGF/activin/nodal signalling pathway is added to the culture medium at a concentration of at least 20 ng/ml, typically comprised between 20 and 70 ng/ml.

Anterior thoracic motor neurons can be obtained by:
1. addition in the culture medium of RA around at least D5 and until around D9, notably from D5 to D9, after the beginning of the culture of hPSCs in the culture medium comprising the activator of the Wnt signalling pathway, or
2. by addition in the culture medium of RA around at least around D3 or D4 and until around D9, notably from D3 to D9 after the beginning of the culture of hPSCs in the culture medium comprising the activator of the Wnt signalling pathway, in combination with the addition of a FGFR agonist, or of an activator of the TGF/activin/nodal signalling pathway (such as GDF11 or GDF8) around 24 to 48 hours before RA addition in the culture medium; optionally wherein the FGFR agonist is added to the culture medium at a concentration of at least 60 ng notably between 60 and 250 ng/ml, between 60 and 200 ng/ml, preferably between 60 and 150 ng/ml, notably at about 120 ng/ml, or wherein the activator of the TGF/activin/nodal signalling pathway is added to the culture medium at a concentration of at least 20 ng/ml, typically comprised between 20 and 70 ng/ml.

Caudal thoracic motor neurons can be obtained by
addition in the culture medium of RA around D3 and until around D9 after the beginning of the culture of hPSCs in the culture medium comprising the activator of the Wnt signalling pathway, combined with the addition in the culture medium at around D3 and until around D4 of a combination of a FGFR agonist and an activator of the TGF/activin/nodal signalling pathway, optionally wherein the FGFR agonist is added at a concentration of at least 100 ng/ml, notably between 100 and 250 ng/ml, between 100 and 200 ng/ml, preferably between 100 and 150 ng/ml, notably at about 120 ng/m; and the activator of the TGF/activin/nodal signalling pathway (such as GDF11 or GDF8) is added to the culture medium at a concentration of at least 20 ng/ml, typically comprised between 20 and 70 ng/ml.

Lumbar motor neurons, including FOXP1 high arm innervating motor neurons, can be obtained by
addition in the culture medium of RA between around D5 and until around D9 after the beginning of the culture of hPSCs in the culture medium comprising the activator of the Wnt signalling pathway, combined with the addition in the culture medium at around D3 or D4 and until D5, notably between D3 and D5 or a combination of a FGFR agonist and an activator of the TGF/activin/nodal signalling pathway (such as GDF11 or GDF8), optionally wherein the FGFR agonist is added at a concentration of at least 100 ng/ml, notably between 100 and 250 ng/ml, between 100 and 200 ng/ml, preferably between 100 and 150 ng/ml, notably at about 120 ng/m; and the activator of the TGF/activin/nodal signalling pathway is added to the culture medium at a concentration of at least 20 ng/ml, typically comprised between 20 and 70 ng/ml.

In a particularly preferred embodiment of the invention, the method for producing a population of spinal motor neuron of a defined rostro-caudal identity comprises the following steps:
1. Generation of axial progenitors: from day 0 (DO), culturing hPSC, notably in the form of human embryoid bodies, in a culture medium comprising at least a Wnt agonist, notably selected from CHIR-9921 or the Wnt3a protein, and optionally an inhibitor of the BMP signalling pathway and an inhibitor of the TGF/activin nodal signalling pathway (for example LDN-193189 and SB431542 respectively), optionally wherein
   the compound CHIR-99021 is added in the culture medium in a concentration ranging from 0.5 to 5 μM, preferably ranging from 1 to 4 μM, even more preferably at about 3 μM,
   the compound LDN193189 is added the culture medium comprising hPSCs in a concentration ranging from 0.05 to 1 μM, preferably from 0.1 to 0.5 μM, from 0.15 to 0.25 μM, even more preferably at about 0.2 μM,
   the compound SB431542 is added to the culture medium comprising hPSCs in a concentration ranging from 10 to 75 μM, preferably ranging from 20 to 50 μM, even more preferably at about 40 μM.
the inhibitor of the BMP signalling pathway and the inhibitor of the TGF/activin/nodal signalling pathway are added to the culture medium for a period of time ranging from 3 to 4 days (i.e. from DO to D3 or from DO to D4), in particular the inhibitor of the TGF/activin/nodal signalling pathway can be added from DO to D3, and
the Wnt activator are added to the culture medium for a period of time ranging from 2 to 4 day, notably from 3 to 4 days, (i.e., from DO to D2, from DO to D3, or from DO to D4).
2. Obtention of spinal motor neurons of a defined rostro-caudal identity:
   a) addition in the culture medium of RA around D3 and until around D9 after the beginning of the culture of hPSCs in the culture medium comprising the activator of the Wnt signalling pathway, and of an agonist of the Hedgehog signalling pathway at a concentration of at least 300 nM, notably between 100 nM and 1 μM, or 100 to 1 μM, preferably ranging from 300 to 750 nM, even more preferably at about 500 nM between day D3 and D9, between day 4 (D4) and day 9 (D9), between D5 and D9, between D6 and D9 or between D7 and D9, in particular between D3 and D9, or
   b) addition in the culture medium of RA around D3 and until around D9 after the beginning of the culture of hPSCs in the culture medium comprising the activator of the Wnt signalling pathway and of an agonist of the Hedgehog signalling pathway at a concentration of at least 300 nM, notably between 100 nM and 1 μM, or 100 to 1 μM, preferably ranging from 300 to 750 nM, even more preferably at about 500 nM between day D3 and D9 or D10 or longer, between day 4 (N4) and day 9 (D9) or D10 or longer, between D5 and D9 or D10 or longer, between D6 and D9 or D10 or longer or between D7 and D9 or D10 or longer, in particular between D3 and D9 or D10 or longer, in combination with the addition of a FGFR agonist or of activator of the TGF/activin/nodal signalling pathway between D3 and D4, optionally wherein the FGFR agonist is added to the culture medium at a concentration of at least 100 ng/ml, notably between 100 and 250 ng/ml, between 100 and 200 ng/ml, preferably between 100 and 150 ng/ml, notably at about 120 ng/ml, and wherein the activator of the TGF/activin/nodal signalling pathway is added to the culture medium at a concentration of at least 20 ng/ml, typically comprised between 20 and 70 ng/ml;
   optionally wherein retinoic acid is added to the culture medium at a concentration ranging from 5 to 1000 nM, preferably ranging from 10 to 500 nM, or ranging from 100 nM to 500 nM. Typically, RA is added to the culture medium at a concentration of at least 100 nM.
to obtain brachial motor neurons, including FOXP1 high innervating motor neurons.
   a′) addition in the culture medium of RA around at least D5 and until around D9, notably from D5 to D9, after the beginning of the culture of hPSCs in the culture medium comprising the activator of the Wnt signalling pathway, and of an agonist of the Hedgehog signalling pathway at a concentration of at least 300 nM, notably between 100 nM and 1 μM, or 100 to 1 μM, preferably ranging from 300 to 750 nM, even more preferably at about 500 nM between day D3 and D9, between day 4 (N4) and day 9 (D9), between D5 and D9, between D6 and D9 or between D7 and D9, in particular between D3 and D9, or
   b′) addition in the culture medium of RA around at least around D3 or D4 and until around D9, notably from D3 to D9 after the beginning of the culture of hPSCs in the culture medium comprising the activator of the Wnt signalling pathway, and of an agonist of the Hedgehog signalling pathway at a concentration of at least 300 nM, notably between 100 nM and 1 μM, or 100 to 1 μM, preferably ranging from 300 to 750 nM, even more preferably at about 500 nM between day D3 and D9, between day 4 (D4) and day 9 (D9), between D5 and D9, between D6 and D9 or between D7 and D9, in particular between D3 and D9, in combination with the addition of a FGFR agonist, or of activator of the TGF/activin/nodal signalling pathway, 24 to 48 hours before RA addition in the culture medium; optionally wherein the FGFR agonist is added to the culture medium at a concentration of at least 60 ng notably between 60 and 250 ng/ml, between 60 and 200 ng/ml, preferably between 60 and 150 ng/ml, notably at about 120 ng/ml, and wherein the activator of the TGF/activin/nodal signalling pathway is added to the culture medium at a concentration of at least 20 ng/ml, typically comprised between 20 and 70 ng/ml,
   optionally wherein retinoic acid is added to the culture medium at a concentration ranging from 5 to 1000 nM, preferably ranging from 10 to 500 nM, or ranging from 100 nM to 500 nM. Typically, RA is added to the culture medium at a concentration of at least 100 nM.
to obtain anterior thoracic motor neurons. including FOXP1 high innervating motor neurons, and FOXP1/SCIP motor neurons
   a′″) addition in the culture medium of RA around D3 and until around D9, after the beginning of the culture of hPSCs in the culture medium comprising the activator of the Wnt signalling pathway, and of an agonist of the Hedgehog signalling pathway at a concentration of at least 300 nM, notably between 100 nM and 1 μM, or 100 to 1 μM, preferably ranging from 300 to 750 nM, even more preferably at about 500 nM between day D3 and D9, between day 4 (N4) and day 9 (D9), between D5 and D9, between D6 and D9 or between D7 and D9, in particular between D3 and D9, combined with the addition in the culture medium at around D3 and until around D4 of a combination of a FGFR agonist and an activator of the TGF/activin/nodal signalling pathway, optionally wherein the FGFR agonist is added at a concentration of at least 100 ng/ml, notably between 100 and 250 ng/ml, between 100 and 250 ng/ml, typically between 100 and 200 ng/ml, or between 100 and 150 ng/ml, notably at about 120 ng/ml; and the activator of the TGF/activin/nodal signalling pathway is added to the culture medium at a concentration of at least 20 ng/ml, typically comprised between 20 and 70 ng/ml,
   optionally wherein retinoic acid is added to the culture medium at a concentration ranging from 5 to 1000 nM, preferably ranging from 10 to 500 nM, or ranging from 100 nM to 500 nM. Typically, RA is added to the culture medium at a concentration of at least 100 nM.
to obtain caudal thoracic motor neurons.
   a″″) addition in the culture medium of RA between around D5 and until around D9 after the beginning of the culture of hPSCs in the culture medium comprising the activator of the Wnt signalling pathway and of an agonist of the Hedgehog signalling pathway at a concentration of at least 300 nM, notably between 100 nM and 1 μM, or 100 to 1 μM, preferably ranging from 300 to 750 nM, even more preferably at about 500 nM between day D3 and D9, between day 4 (D4) and day 9 (D9), between D5 and D9, between D6 and D9 or between D7 and D9, in particular between D3 and D9, combined with the addition in the culture medium at around D3 or D4 and until D5, notably between D3 and D5 or a combination of a FGFR agonist and an activator of the TGF/activin/nodal signalling pathway, optionally wherein the FGFR agonist is added at a concentration of at least 100 ng/ml, notably between 100 and 250 ng/ml, between 100 and 200 ng/ml, or between 100 and 150 ng/ml, notably at about 120 ng/ml; and the activator of the TGF/activin/nodal signalling pathway is added to the culture medium at a concentration of at least 20 ng/ml, typically comprised between 20 and 70 ng/ml,
   optionally wherein retinoic acid is added to the culture medium at a concentration ranging from 5 to 1000 nM, preferably ranging from 10 to 500 nM, or ranging from 100 nM to 500 nM. Typically, RA is added to the culture medium at a concentration of at least 100 nM.
to obtain lumbar motor neurons, including FOXP1 high arm innervating motor neurons.
3. Optionally differentiation of spinal motor neurons progenitors into post-mitotic spinal motor neurons: around 9 days after the beginning of the culture of hPSCs in the culture medium comprising the activator of the Wnt signalling pathway, addition of an inhibitor of the Notch signalling pathway, typically DAPT for a period of time of at least 5 to 7 days or more, typically for 5 days, optionally wherein DAPT is present in the culture medium in a concentration ranging from 1 to 25 μM, more preferably ranging from 5 to 20 μM, notably at about 10 μM.

A representative scheme of an advantageous method for obtaining spinal motor neurons of a defined rostro-caudal identity is notably provided in FIG. 4A.

Culture Mediums

In the context of the invention the term “culture medium” refers to a liquid medium suitable for the in vitro culture of mammalian cells. The culture media used in the methods of the invention may be based on a commercially available medium such as DMEM/F12 from Invitrogen or a mixture of DMEM/F12 and Neurobasal in a 1:1 ratio, from Invitrogen.

The culture media used in the methods of the invention may also comprise various supplements such as B27 supplement (Invitrogen) and N2 supplement (from Invitrogen).

The B27 supplement contains, amongst other constituents, SOD, catalase and other anti-oxidants (GSH), and unique fatty acids, such as linoleic acid, linolenic acid, lipoic acids.

The culture media used in the methods of the invention can in particular be based on a N2B27 medium.

The term “N2B27” refers to the medium described in Liu et al. (2006) Bioc em. Biop ys. Res. Commun. 346:131-139, which comprises DMEM/F12 and Neurobasal media in a 1:1 ratio, N2 supplement (1/100), B27 supplement (1/50) and β-mercaptoethanol (1/1000).

Preferably, the culture media used in the methods of the invention further comprise the ROCK inhibitor Y-27632, typically at a concentration of about 5 μM.

In the methods of the invention, if necessary, the culture media can be renewed, partly or totally, at regular intervals. Typically, the culture media can be replaced with fresh culture media every other day, for the periods of time mentioned below.

In some embodiments, the present invention also encompasses

A culture medium suitable for obtaining brachial motor neurons including FOXP1 high innervating motor neurons comprising at least:
an agonist of the Hedgehog signalling pathway at a concentration of at least 300 nM, notably between 100 nM and 1 μM, or 100 to 1 μM, preferably ranging from 300 to 750 nM, even more preferably at about 500 nM,
a FGFR agonist, optionally wherein the FGFR agonist is added to the culture medium at a concentration of at least 100 ng/ml, notably between 100 and 250 ng/ml, between 100 and 200 ng/ml, preferably between 100 and 150 ng/ml, notably at about 120 ng/ml or an activator of the TGF/activin/nodal signalling pathway (e.g., GDF11 or GDF8) at a concentration of at least 20 ng/ml, typically comprised between 20 and 70 ng/ml, and
retinoic acid at a concentration ranging from 5 to 1000 nM, preferably ranging from 10 to 500 nM, or ranging from 100 nM to 500 nM. Typically, RA is added to the culture medium at a concentration of at least 100 nM.A

A culture medium suitable for obtaining anterior thoracic motor neurons comprising at least:
an agonist of the Hedgehog signalling pathway at a concentration of at least 300 nM, notably between 100 nM and 1 μM, or 100 to 1 μM, preferably ranging from 300 to 750 nM, even more preferably at about 500 nM
a FGFR at a concentration of at least 60 ng notably between 60 and 250 ng/ml, between 60 and 200 ng/ml, preferably between 60 and 150 ng/ml, notably at about 120 ng/ml or an activator of the TGF/activin/nodal signalling pathway (e.g., GDF11 or GDF8) at a concentration of at least 20 ng/ml, typically comprised between 20 and 70 ng/ml,
retinoic acid at a concentration ranging from 5 to 1000 nM, preferably ranging from 10 to 500 nM, or ranging from 100 nM to 500 nM. typically, at a concentration of at least 100 nM.

A culture medium suitable for obtaining caudal thoracic motor neurons comprising at least:
an agonist of the Hedgehog signalling pathway at a concentration of at least 300 nM, notably between 100 nM and 1 μM, or 100 to 1 μM, preferably ranging from 300 to 750 nM, even more preferably at about 500 nM
a FGFR agonist is added at a concentration of at least 100 ng/ml, notably between 100 and 250 ng/ml, between 100 and 200 ng/ml, preferably between 100 and 150 ng/ml, notably at about 120 ng/m; and the activator of the TGF/activin/nodal signalling pathway is added to the culture medium at a concentration of at least 20 ng/ml, typically comprised between 20 and 70 ng/ml
retinoic acid at a concentration ranging from 5 to 1000 nM, preferably ranging from 10 to 500 nM, or ranging from 100 nM to 500 nM, typically, at a concentration of at least 100 nM.

A culture medium suitable for obtaining lumbar motor neurons, including FOXP1 high arm innervating motor neurons comprising at least:
an agonist of the Hedgehog signalling pathway at a concentration of at least 300 nM, notably between 100 nM and 1 μM, or 100 to 1 μM, preferably ranging from 300 to 750 nM, even more preferably at about 500 nM
a FGFR agonist and an activator of the TGF/activin/nodal signalling pathway, wherein the FGFR agonist is at a concentration of at least 100 ng/ml, notably between 100 and 250 ng/ml, between 100 and 200 ng/ml, preferably between 100 and 150 ng/ml, notably at about 120 ng/m; and the activator of the TGF/activin/nodal signalling pathway at a concentration of at least 20 ng/ml, typically comprised between 20 and 70 ng/ml,
retinoic acid at a concentration ranging from 5 to 1000 nM, preferably ranging from 10 to 500 nM, or ranging from 100 nM to 500 nM, typically at a concentration of at least 100 nM.

The present invention will be further illustrated by the following figures and examples.

### BRIEF DESCRIPTION OF THE FIGURES

FIG. 1: Aging hPSC-derived axial progenitors generate progressively more caudal motor neuron subtypes

(A) Schematic summary of data in FIG. S1. MNs defined by the expression of ISL1 or HB9 (here in grey) are organized in motor columns in spinal ventral horns. HOX expression profiles within MNs and localization of MNs expressing high level of FOXP1 and SCIP are represented as observed in the human embryonic spinal cord at 6.3 and 7.5 weeks of gestation. Changes in shapes indicate an increase or decrease in the number of MNs expressing a given marker. FOXP1^{high} MNs are observed selectively in lateral motor columns (LMC) of the brachio-thoracic and lumbar spinal cord. SCIP^{high} MNs are a subset of LMC MNs in the caudal brachial spinal cord. (B) Differentiation conditions used in C to F in which the time of exposure to RA/SAG is modulated. (C) Immunostaining for ISL1, HB9 (MNs), NEFL (neurons), HOX transcription factors, FOXP1 and SCIP on cryostat sections of EBs on day 14 of differentiation. The later RA is applied the more caudal MNs are. FOXP1 and SCIP MNs are mostly generated in RA D4 and D5 further defining the rostro-caudal identity of the MNs within the HOXC8+ conditions. Scale bar: 100 μm. (D-F) Proportion of MNs (ISL1+ cells) expressing the indicated markers. Data are shown as mean±SD. Each circle is an independent experiment. * if P≤0.05, ** if P≤0.01 and *** if P≤0.001. See also FIGS. S1 and S2.

FIG. 2: Temporal transcriptomic analysis of hPSC-derived axial progenitors.

(A) Experimental design. (B) 20 most enriched genes in D2 progenitors versus hESC. Progenitors acquired a transcriptomic signature similar to the one of murine axial progenitors. (C) Fold enrichment in D3 progenitors versus hESC of the 20 most enriched neuromesodermal progenitor (NMP) genes defined in (Gouti et al., 2017). (D) Immunostaining for axial progenitor markers on hiPS-derived D2 and D3 progenitors (WTS2 line). Cells co-express the three markers. Scale bars: 40 μm. (E) Temporal transcriptional changes in HOX genes coding for HOX regionally expressed in human MNs in vivo. (F) Reactome pathway analysis of the 232 genes upregulated 2-fold (p-value<0.05) between D3 and D2. Y axis: FDR=false discovery rate. X axis=enrichment score calculated for a given Reactome pathway. (G) Temporal evolution of upregulated MAPK target genes and FGF ligands in aging axial progenitors. ETV5 is a transcription factor activated by ERK1/2 MAPK in many other systems. The other genes encode feedback negative regulators of MAPK pathways. All data are shown as mean±SD. (H) Schematic representation of the transcriptional and immunostaining analysis of day 2 and 3 progenitors.

FIG. 3: FGF pathway inhibition stalls HOX temporal induction and caudal MN specification

(A) Differentiation conditions. PD173074 (FGFR1-3 inhibitor) or PD032590 (MEK1/2 inhibitor) were added at D3 up to D7. (B) Proportion of MNs (ISL1+ cells) in the different conditions (mean±SD). (C) Immunostaining for ISL1 (MNs) and HOX transcription factors on cryostat sections of embryoid bodies on day 14 of differentiation. MEK and FGFR inhibitors prevent the specification of HOXC8 and HOXC9 MNs. Instead, HOXA5, HOXC6 MNs are generated. Scale bar: 100 μm. (C) Quantification of HOXC6 and HOXC9 MNs on day 14 of differentiation. (D) Real time PCR analysis of HOX mRNAs in progenitors at D3, D4 and D5 of differentiation. MEK and FGFR inhibitors prevent the temporal increase in caudal HOX expression. Data are shown as mean±SD; Each circle is an independent experiment. * if P≤0.05, ** if P≤0.01 and *** if P≤0.001.

FIG. 4: Dynamic pacing of HOX induction and specification of discrete MN subtypes upon changes in FGF2 and GDF11 levels.

(A) Differentiation conditions. Extrinsic cues, FGF2, GDF11, or FGF2+GDF11 were added on day 3 of differentiation at various concentrations or for different durations (B) Immunostaining for HOX on cryostat sections of embryoid bodies on day 14 of differentiation. FGF2, GDF11 and FGF2/GDF11 induce more caudal MN subtypes. Scale bar: 100 μm. (C, D, E) Proportion of MNs (ISL1+ cells) expressing the indicated markers. The effect of the duration of FGF2 treatment (C), FGF2 concentration (D) and duration of GDF11 or FGF2+GDF11 (E) were monitored. (F) Real time quantitative PCR analysis of the expression of HOX genes regionally expressed in human MNs in vivo. HOX mRNAs were monitored at D4 (24 h post-treatment) and D5 (48 h post-treatment) upon addition of FGF2, GDF11 or a combination of FGF2 (120 ng/ml) together with GDF11 (25 ng/ml). Data is normalized at each time point to the control condition treated with only RA at day 3. Data are shown as mean±SD. Each circle is an independent experiment. * if P≤0.05, ** if P≤0.01 and *** if P≤0.001. See also FIG. 5.

### RESULTS

Materials and Methods

Human Embryonic Spinal Cord Histology

Human fetal embryo of 6.3 weeks of gestation were obtained from pregnant women referred to the Department of Gynecology and Obstetrics at the Antoine Béclère hospital (Clamart, France) for legally induced abortions in the first trimester of pregnancy as previously described (Lambrot et al., 2006). All women provided written informed consent for scientific use of the fetal tissues. None of the abortions were due to fetal abnormality. The fetal age was determined by measuring the length of limbs and feet (Evtouchenko et al., 1996). The project was approved by the local Medical Ethics Committee and by the French Biomedicine Agency (reference number PFS 12-002). Alternatively, Human embryonic spinal cords (n=2) at stage 7.5 weeks of gestation were collected in accordance with the national guidelines of the United States (National Institutes of Health, U.S. Food and Drug Administration) and the State of New York and under Columbia University institutionally approved ethical guidelines relating to anonymous tissue. The material was obtained after elective abortions and was classified on the basis of external morphology according to the Carnegie stages. Gestational age was determined by last menstrual period of the patient or by ultrasound, if the ultrasound estimate differed by 1 week as indicated by the obstetrician. In all cases, the spinal cord was removed as intact as possible before fixation with fresh, cold 4% PFA for 1.5 h on ice washed abundantly with PBS and then cryoprotected overnight in 30% sucrose. Post-fixation, the cord was measured and cut into anatomical sections to accommodate embedding in OCT Compound (Leica) and stored at −80° C. before cutting on a cryostat. Sections (16 μm) were cut along the full length of the cord.

Human Pluripotent Stem Cell Lines

Human SA001 embryonic stem cell (ESC) line (male, RRID: CVCL_B347) was obtained from Cellectis and used accordingly to the French current legislation (Agency of Biomedicine, authorization number AFSB1530532S). Induced pluripotent stem cell (iPSC) line WTSIi002 (male, RRID: CVCL_AH30, alternative name HPSIO913i-eika_2) was obtained from the European Bank for Pluripotent Stem Cells (EBISC). WTC-mEGFP-Safe harbor locus (AAVS1)-c16 produced by the Allen cell institute was obtained from Coriell (Cat #AICS-0036-006, male, RRID:CVCL_JM19). were obtained from the European Bank for Pluripotent Stem Cells (EBISC). Experiments with iPSCs were approved by relevant ethic committees (declaration DC-2015-2559). All PSC lines were cultured at 37° C. on Matrigel (Corning) in mTSER1 medium (Stem Cell Technologies) and amplified using EDTA (Life Technologies) clump-based passaging. They were tested for potential mycoplasma contamination every other week (MycoAlert™ Mycoplasma Detection Kit, Lonza, LT07-118). No contamination was detected during the study. PSC were thawed in presence of Y-27632 (10 μM, Stemgent or Stem Cell Technologies) and the culture media was changed every day.

Human Pluripotent Stem Cell Differentiation

Human PSC embryoid body-based differentiation was performed as previously described (Maury et al., 2015). hPSC were dissociated with cold Accutase (Life Technologies) for 3 to 5 min at 37° C. and resuspended in differentiation medium N2B27 (Advanced DMEM F12, Neurobasal vol:vol (Life Technologies)), supplemented with N2 (Life Technologies), B27 without Vitamin A (Life technologies), penicillin/streptomycin 1%, β-Mercaptoéthanol 0.1% (Life Technologies), with Y-27632 (10 μM, Stemgent or Stem Cell Technologies), CHIR-99021 (3 μM or 4 μM Selleckchem) SB431542 (20 μM, Selleckchem) and LDN 193189 (0.1 μM, Selleckchem). 2×10⁵ cells ml⁻¹ were seeded in ultra-low attachment 6 well plates (Corning) to form embryoid bodies (EBs). All conditions of differentiation received the same medium at day 2 and at day 3 but SB431542 was removed at day 3. Then, the differentiation proceeded according to the schemas presented above the figures. SAG (Smoothened Agonist, Merck millipore), FGF2 (Recombinant Human FGF basic, Peprotech), RA (Sigma-Aldrich), GDF11 (Recombinant Human/Murine/Rat GDF11, Peprotech), PD0325901 (Selleckchem), PD173074, SCH772984 (Selleckchem), DAPT (Stemgent) were added at indicated time points. For concentrations see Table S2. Media were changed every other day unless specified.

Embryoid Body Processing for Immunostaining

EBs were collected, rinsed with PBS then fixed with 4% PFA for 5 min at 4° C. and rinsed with PBS 3 times for 5 min. EBs were cryoprotected with 30% sucrose and embedded in OCT (Leica) prior sectioning with a cryostat. Alternatively, day 2, 3 and 4 progenitors were plated on Matrigel (Corning, diluted according to manufacturer recommendation) coated coverslips and let to adhere between for 30 min prior fixation with 4% PFA for 5 min at 4° C.

Immunostaining

All immunostainings were performed as follow: cells or sections were incubated with a saturation solution (PBS/FBS 10%/0.2% Triton) for 10 minutes. Primary antibodies (Table S3) were diluted in staining solution (PBS/2% FBS/0.2% Triton) and incubated overnight at 4° C. in a humidified chamber. Following 4 PBS washes (10 min each), secondary antibodies (Alexa488, Alexa568 and Alexa647, Life Technologies, 1:1,000) were added for 1 h at RT. After 3 PBS washes, DAPI was added on the cells (Invitrogen, 1:3,000) for 5 min. Cells or slices were then mounted in Fluoromount (Sigma Aldrich or Cliniscience).

Image Acquisition

Samples were visualized and imaged using either a ZEISS LSM 880 Confocal Laser Scanning Microscope (Carl Zeiss Microscopy) controlled by Zen black software (Zeiss), a confocal microscope TCS SP5 II (Leica) or a DM6000 microscope (Leica) equipped with CoolSNAP EZ CDD camera, controlled by MetaMorph software (MetaMorph Inc). Alternatively, images were acquired using the automated microscope Cell Discoverer 7 (Zeiss), equipped with an Axiocam 506 m camera, with Zen black software (Zeiss).

Quantitative RT-PCR Analysis

Total RNA were extracted (RNAeasy Plus Mini Kit, Qiagen) and cDNA synthesized using SuperScript III (Invitrogen). Quantitative real-time PCR was performed using a 7900HT fast real time PCR system (Applied Biosystems) with Sybr Green PCR Master Mix (Applied Biosystems). Alternatively performed using QuantStudio 5 Real-Time PCR System (Thermofisher Scientific) and a mix with qPCR Brilliant II SYBR MM with low ROX (Agilent). Primers are listed in Supplementary Table 3. All expression data were normalized to Cyclophilin A mRNA. All analyses were performed with three technical replicates per plate. Relative expression levels were determined by calculating 2-ΔΔCt.

Transcriptomic Analysis

hESC were collected post dissociation and prior exposure to differentiation medium. Progenitors were collected on day 2, day 3 and day 4 of differentiation. For each of the 8 samples, total RNA were extracted then reverse transcribed using the Ion AmpliSeq Transcriptome Human Gene Expression kit (Thermofisher Scientific). The cDNA libraries were amplified and barcoded using Ion AmpliSeq Transcriptome Human Gene Expression core panel and Ion Xpress Barcode Adapter (Thermofisher Scientific). The amplicons were quantified using Agilent High Sensitivity DNA kit before the samples were pooled in sets of eight. Emulsion PCR and Enrichment was performed on the Ion OT2 system Instrument using the Ion PI Hi-Q OT2 200 kit (Thermofisher Scientific). Samples were loaded on an Ion PI v3 Chip and sequenced on the Ion Proton System using Ion PI Hi-Q sequencing 200 kit chemistry (200 bp read length; Thermofisher Scientific). The Ion Proton reads (FASTQ files) were imported into the RNA-seq pipeline of Partek Flow software (v6 Partek Inc) using hg19 as a reference genome. The number of reads per sample was ranging from 7.5 million to 12 million reads. To determine genes that are differentially expressed between groups mapped reads were quantified using Partek E/M algorithm normalized by the Total count/sample (the resulting counts represent the gene expression levels on reads/millions for over 20,800 different genes present in the AmpliSeq Human Gene Expression panel). The evaluation of the differential expression between two conditions was performed using the EdgeR package under R. Pathway enrichment analyses were performed on upregulated genes (FC≥2.0, p-value<0.05) between two time points by interrogating Reactome database. Significant enrichments were calculated using hypergeometrical test and Benjamini-Hochberg correction for multiple comparisons. The enrichment score was calculated as described in (Wang et al., 2017).

The normalized transcriptomic data are provided in Table S1. Raw data are available upon request.

Quantification and Statistical Analysis

All statistics were computed using Graphpad Prism software. One-way analysis of variance (ANOVA) with a Kruskall-Wallis post hoc analysis were performed following normality tests provided by Prism. Number of n, dispersion measures and P-values are indicated in figure legends. In all figures, n are independent differentiations started from independent newly thawed hPSCs vials. For each condition at least 4 independent EBs were imaged in which all the cells were quantified by automated image analysis. The images were exported and saved as TIFF with Fiji if needed (Schindelin et al., 2012). Quantitative analyses on images were performed using the CellProfiler software (Carpenter et al., 2006) (Broad Institute open source at www.cellprofiler.org). DAPI stained nucleus were segmented into primary objects using CellProfiler segmentation pipeline and the nuclear mask was used to define objects on the target channels. The threshold to define positive nuclei for a given target was obtained using EBs' section negative for the target of interest. All images across conditions were then automatically analyzed in batch ensuring unbiased analysis. The analyses of the FOXP1 and SCIP immunostaining intensity were performed combining CellProfiler with the software FCS express 7 (DeNovo Software, Glendale, CA, USA). Nuclei were segmented into primary objects as described above and FOXP1 and SCIP fluorescence intensities were calculated in each primary object. Fluorescence intensity plots for FOXP1 and SCIP were then generated using FCS express 7 software to visualize the intensity levels of the different markers for each individual cells and determine the percentage of cells above a given threshold. Cell profiler pipelines for quantification are available upon request.

Results

HOX Expression Profiles and Motor Neuron Subtypes in Human Embryonic Spinal Cord

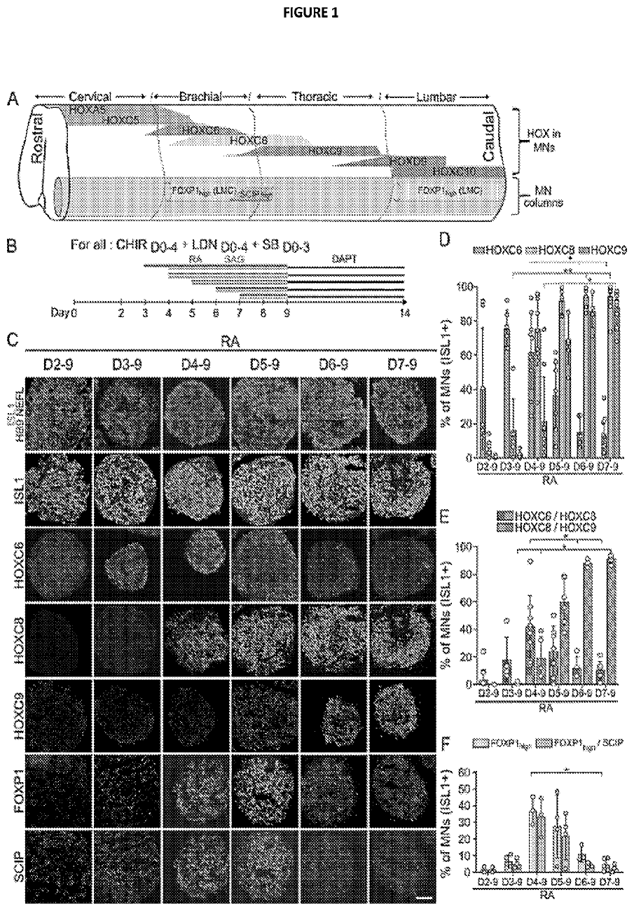

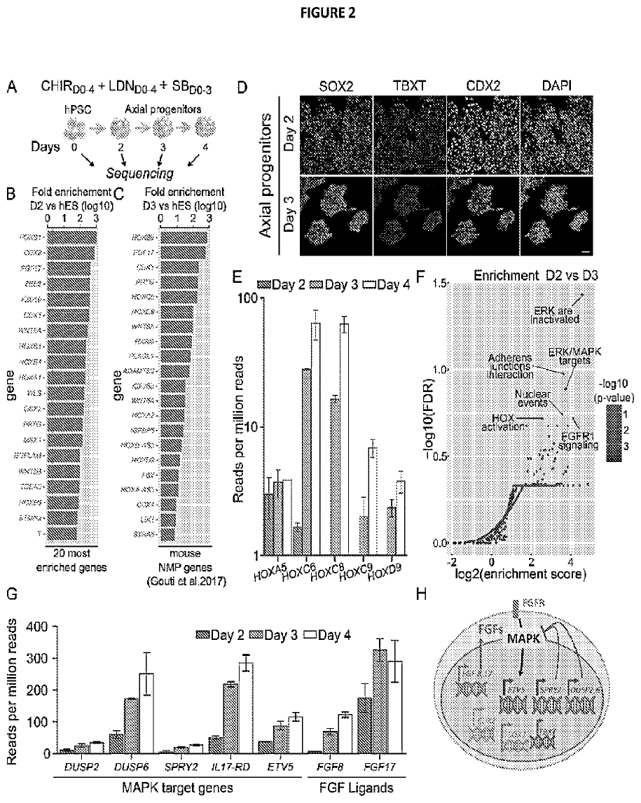

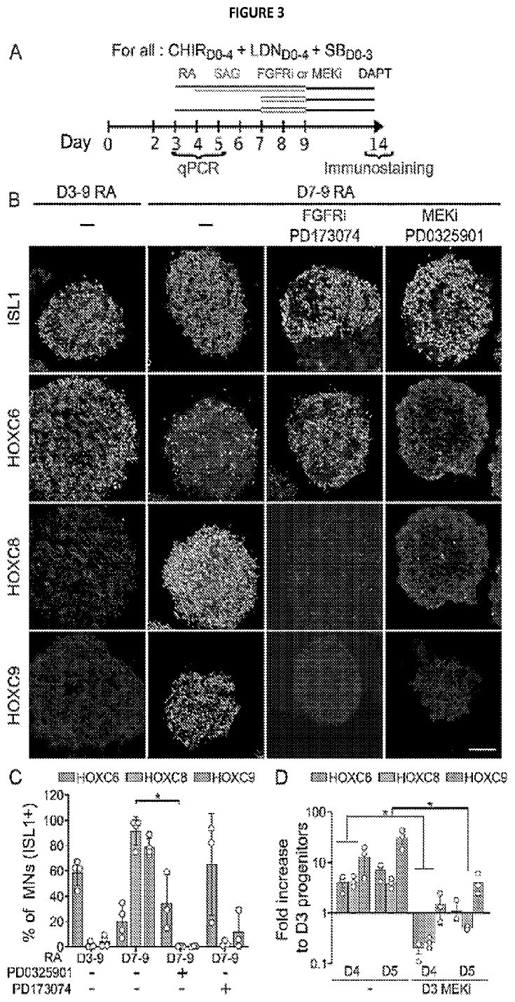

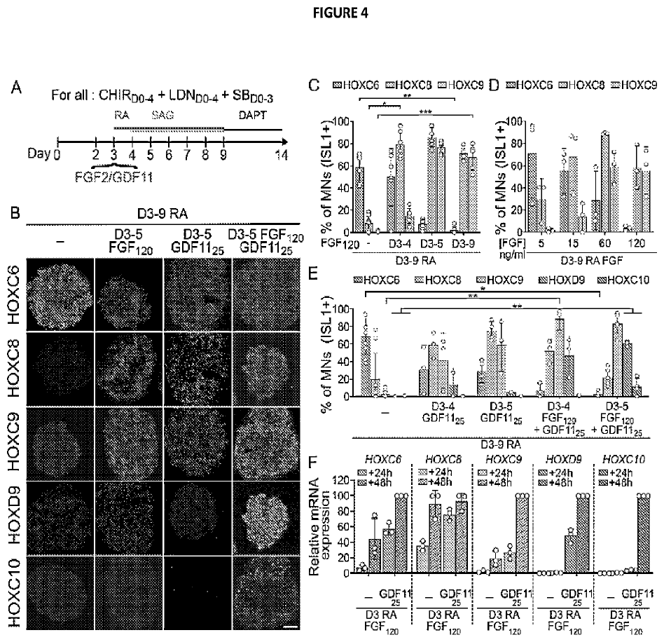

The differential expression of HOX transcription factors along the vertebrate spinal cord is a major product of HOX gene regulation. In spinal motor neurons, this Hox code orchestrates the specification of subtype specific features controlling the formation of locomotor circuits (Dasen, 2017; Philippidou and Dasen, 2013). Whether the spinal HOX code and associated MN subtypes is conserved in human remains unknown preventing faithful assessment of HOX regulation and its link with cell “rostro-caudal” identity during hPSC differentiation. We thus mapped, in human embryos at 6.3 and 7.5 weeks of development, 7 HOX transcription factors that display collinear expression patterns and instruct MN subtype specification in mouse and chick (Philippidou and Dasen, 2013) (FIG. 1A). As in animal models, human MNs expressed ISL1 or HB9 all along the spinal cord (FIG. 1A) (Amoroso et al., 2013). Within MNs, HOX displayed rostro-caudal patterns resembling those of mouse and chick (Dasen et al., 2003; Liu et al., 2001): cervical MNs expressed HOXA/C5, while brachial MNs expressed HOXC6, thoracic MNs HOXC9 and lumbar MNs HOXC10. Caudal brachial MNs co-expressed HOXC6 and HOXC8 and anterior thoracic ones HOXC8 and HOXC9. HOXD9 labeled caudal thoracic MNs as well as anterior lumbar MNs together with HOXC10 (FIG. 1A). In amniotes, this Hox code instructs the formation of distinct motor columns that innervate common muscle groups, and motor pools that innervate a single muscle (Philippidou and Dasen, 2013). To be able to assess in vitro whether changes in HOX expression result in the specification of appropriate neuronal subtypes, we mapped MN subtype markers in regard to HOX expression domains (FIG. 1A). As in mouse and chick, MNs expressing high level of FOXP1 (FOXP1^{high}) were observed at brachio-thoracic (HOXC6 and HOXC8/HOXC9) and lumbar (HOXC10) levels (FIG. 1A). They formed a lateral motor column (LMC) where limb innervating MNs are located (Amoroso et al., 2013; Routal and Pal, 1999). Within this FOXP1^{high} LMC, SCIP/HOXC8 MNs were observed in the caudal brachial spinal cord. In contrast, SCIP/HOXC8/HOXC9 MNs were located in the anterior thoracic region. Their location and their transcriptional code identify them as putative hand-controlling MNs (Bell et al., 2017; Mendelsohn et al., 2017).

Overall, HOX transcription factors are regionally expressed along the rostro-caudal axis of the human spinal cord. Within these different HOX domains, distinct MN subtypes, identifiable by combination of transcription factors, are generated at stereotyped positions. This data provided readouts to assess the mechanisms regulating HOX expression in axial progenitors and their impact on cell type specification during hPSC differentiation.

Aging Axial Progenitors Generate More Caudal Neuronal Subtypes

We first sought to test whether axial progenitors (i.e. progenitors competent to generate caudal cell types of distinct rostro-caudal identities) could be generated using 3D differentiation of human embryonic stem cells (hESC) by monitoring MN subtype specification. We previously reported sequences of extrinsic cues leading to the targeted generation of spinal MNs from human PSCs through a putative axial progenitor stage (Maury et al., 2015). Exposure of embryoid bodies (EBs) to a Wnt pathway agonist (CHIR99021) and inhibitors of BMP and TGFβ pathways (SB431542 and LDN193189) generated progenitors that expressed CDX2, a marker of axial progenitors and a regulator of caudal HOX gene induction (Bel-Vialar et al., 2002; Bialecka et al., 2010; Gouti et al., 2014; Maury et al., 2015; Mazzoni et al., 2013; Neijts et al., 2017). Spinal MNs (70% of the cells in average) were generated upon exposure, at day 2 or 4, to Retinoic acid (RA) and an agonist of the sonic hedgehog pathway (SAG) which respectively promotes neurogenesis and guides axial progenitors towards a ventral MN fate (Briscoe and Novitch, 2008; Maury et al., 2015; Ribes et al., 2009). Here, we assessed the rostro-caudal identity of MNs produced in these conditions (FIG. 1B). Staining for HB9, ISL1 and the pan-neuronal marker neurofilament light chain (NEFL) together with quantification of ISL1+ cells, confirmed the efficient generation of spinal MNs as previously shown (Maury et al., 2015) (FIG. 1C). Analysis of HOX expression showed that RA/SAG from Day 2 (D2) up to D9 gave rise to HOXC6 MNs corresponding to anterior brachial MNs; an identity acquired by most MNs following addition of RA/SAG from D3 to 9 (74.6% of HOXC6, 17.3% HOXC8 MNs) (FIG. 1D, E). Addition of RA/SAG from D4 to 9 generated MNs with a caudal brachial identity (41.8% HOXC6/C8) from which 37.1% expressed high level of FOXP1 corresponding to limb-innervating MNs in the spinal cord (FIG. 1D-F). Overall, these results suggested that MN subtype identity was dependent on either i) the duration of exposure to RA; with a shorter RA exposure promoting caudalization or ii) the time at which they received RA as previously suggested (Bel-Vialar et al., 2002; Del Corral and Storey, 2004; Lippmann et al., 2015). To distinguish between these possibilities, D3 progenitors were exposed to reduced duration of RA (D3-5, D3-8 versus D3-9). None of these shorter RA treatments promoted the birth of more caudal MNs. These results showed that the day at which progenitors are exposed to RA/SAG is the main trigger of caudalization. Further delaying RA/SAG induced even more caudal MN subtypes. On D5, it generated MNs with an anterior thoracic identity (59.8% HOXC8/C9) (FIG. 1B-E) from which 27.6% MNs acquired a FOXP1+ limb-innervating identity (FIG. 1F). HOXC9/FOXP1/SCIP, which are located in the human anterior thoracic spinal cord and might correspond to hand-innervating MNs, were observed almost exclusively in this condition (FIGS. 1F, S2F-G). Then RA/SAG (D6 or 7) specified MNs acquiring a mid-thoracic identity as demonstrated by the expression of HOXC9 and the loss of FOXP1^{high} MNs (FIGS. 1B-F, S2F-G). The progressive caudalization of MN identity upon incremental delays of RA/SAG addition was confirmed with an induced PSC (iPSC) line. Importantly, an increased concentration of CHIR (from 3 to 4 μM) was necessary to generate a homogenous population of progenitors expressing CDX2 on D3 and to observe the subsequent specification of caudal MN subtypes (FIGS. S2H-J). This suggests line to line differences, a result of importance for future studies.

Overall, Wnt activation combined to TGF-□/BMP pathway inhibition converts hPSC into progenitors competent to generate progenies expressing distinct HOX combinations and t found at different rostro-caudal positions in human embryos. Hence, these progenitors qualify as axial progenitors. The duration of the time window between Wnt and RA establishes the final positional identity of the progenies.

Parallel Induction of Caudal HOX Genes and FGF Target Genes in Aging Axial Progenitors

Then, we sought to determine the molecular changes occurring over time in aging axial progenitors, prior to RA exposure. We performed a comparative transcriptomic analysis of hESC and hESC-derived axial progenitors on day 2, 3 and 4 of differentiation (FIG. 2A and Table S1). Pathway analysis of the genes enriched more than 2 fold (p<0.05) in the progenitors compared to hESC indicated an activation of the Wnt pathway paralleled by a transcriptional activation of HOX genes (not shown). In agreement with their axial potential, D2, D3 and D4 progenitors showed a high enrichment in transcripts characterizing the cells of the mouse caudal lateral epiblast in which axial progenitors reside (Expression of CDX1 and 2, TBXT (BRACHYURY), FGF17, RXRG, SP5/8, WNT5A/B, WNT8A, FGF8, GRSF1, CYSTM1, HES3 together with SOX2); a loss of most pluripotency markers and the absence of typical markers of node cells, mesodermal and allantois (FIG. 2B-C and Table S1) (Cambray and Wilson, 2007; Edri et al., 2019; Gouti et al., 2014; Gouti et al., 2017; Henrique et al., 2015; Koch et al., 2017; Wymeersch et al., 2016; Wymeersch et al., 2019). Furthermore, their transcriptome is highly similar to the one of mouse bipotent neuromesodermal progenitors (NMPs), which represent a population of axial progenitors (Gouti et al., 2014; Gouti et al., 2017; Henrique et al., 2015; Tzouanacou et al., 2009; Wymeersch et al., 2016). Among the 142 genes defining early and late NMPs (Gouti et al., 2017), the D2, D3 or D4 progenitors expressed 122 of them which included the most enriched genes when comparing progenitors to hESC (FIG. 2C). Accordingly, D2 and D3 axial progenitors co-expressed SOX2, CDX2 and low level of TBXT at protein level (FIG. 2D), a signature associated with NMPs (Attardi et al., 2018; Denham et al., 2015; Diaz-Cuadros et al., 2020; Edri et al., 2019; Faustino Martins et al., 2020; Gouti et al., 2014; Gouti et al., 2017; Lippmann et al., 2015; Metzis et al., 2018; Olivera-Martinez et al., 2012; Wymeersch et al., 2019). Overall, these results demonstrated that Wnt-induced hPSC-derived axial progenitors resemble axial progenitors of the mouse anterior caudal lateral epiblast that feed the elongation of the spinal cord (Edri et al., 2019; Gouti et al., 2014; Gouti et al., 2017; Henrique et al., 2015; Koch et al., 2017; Liu et al., 2001; Wymeersch et al., 2016).

Then, comparative analysis of the transcriptome of aging progenitors indicated a temporal collinear activation of HOX genes that display regionalized expression patterns in the spinal cord. At D2, the progenitors expressed HOXA5 and low level of HOXC6, which belongs to the 3′ half of HOX complexes with anterior borders of expression in the anterior spinal cord (FIG. 1A, Si). On D3, HOXC8 was well expressed and HOXC9 expression had started, two genes belonging to the center part of HOXC complex with expression in the middle of the spinal cord (FIGS. 1A, S1). Their expression further increased at day 4 (3.8 and 3.4-fold respectively) in progenitors that will give rise to HOXC8 and some HOXC9 MNs when exposed to RA/SAG (FIG. 1C-D). Non-expressed HOX genes corresponded to the 10 to 13 paralog groups, the latest and most caudally expressed HOX (Gaunt, 1991; Izpisda-Belmonte et al., 1991; Philippidou and Dasen, 2013) and FIG. 1A). The temporality of the collinear activation in axial progenitors is thus in agreement with the generation of more caudal MNs when RA/SAG was delayed (FIG. 1). Hence, Wnt induced a temporal collinear activation of HOX genes, which parallels the change in rostro-caudal potential of the progenitors.

To define the pathways activated in parallel with the sequential induction of HOX genes, we performed a pathway analysis on the genes increased more than 2 fold (p<0.05) between D2 and D3. Among the 232 genes, we detected an enrichment for annotations associated with an activation of the Mitogen-activated protein kinases (MAPK) pathways due to a gradual increase in expression of typical MAPK target genes (ETV5, DUSP4, DUSP6, IL17DR or SPRY2) (FIG. 2F-H). MAPKs are classical mediators of FGF signaling (Lunn et al., 2007). In agreement, together with the rise in MAPK target genes we observed an increase in FGF8 and FGF17 expression, two secreted FGF ligands previously described to increase over time in the caudal epiblast of chick embryos (FIG. 2G-H) (Liu et al., 2001; Wymeersch et al., 2019).

Hence, the sequential collinear expression of HOX genes is paralleled by an increase in expression of FGF ligands and MAPK target genes within axial progenitors. As FGFs promote caudal Hox genes in other systems (Bel-Vialar et al., 2002; Dasen et al., 2003; Liu et al., 2001), we postulated that paracrine or autocrine FGF signaling might be triggering the sequential induction of HOX genes.

FGF Signaling is Required for HOX Sequential Activation and Caudal MN Specification

We aimed at testing whether endogenous FGF signaling was necessary for the temporal shift in axial progenitor rostro-caudal potential and HOX gene induction. For that, we blocked FGF signaling in aging progenitors prior RA exposure and tested the impact on caudal HOX gene induction and MN subtype specification. We exposed progenitors from D3 to D7 to i) PD173074, a selective FGFR1/3 antagonist or ii) PD0325901 which inhibits the MAPK kinase MEK1/2 (FIG. 3A). The efficiency of MN generation was not impacted by the two inhibitors, even though we collected a reduced number of EBs in PD173074 condition (FIG. 3B). In control condition, RA/SAG at D7 induced MNs expressing HOXC8 (97.5%) and C9 (87.6%). In contrast, addition of the inhibitors at D3 while adding RA/SAG at D7 generated MNs with an anterior brachial identity (HOXC6). This more anterior identity was normally obtained when RA was added on early D3 progenitors showing that the inhibitors blocked the temporal change in rostro-caudal potential (FIG. 3A-C). We then tested whether the dissociation between the age of the progenitors and the rostro-caudal identity of progeny was linked to a stalled sequence of HOX gene induction in axial progenitors. We monitored the expression of HOXC6, C8 and C9 mRNAs following MEK1/2 inhibition at D3. While in control condition, the three genes increased over time, MEK inhibition blocked this increase at D4 or D5 (FIG. 3D). Hence, inhibition of FGF signaling in aging axial progenitors blocks HOX temporal induction and caudal cell type specification. Our transcriptomic analysis indicated a temporal increase in FGF8 and FGF17 expression suggesting that an increase in FGF concentration and/or duration of exposure might be pacing the HOX clock.

FGF Level Paces the HOX Clock and MN Subtype Specification

To test whether the level of environmental FGF paces the HOX clock, we exposed early D3 progenitors, to RA/SAG together with FGF2 at different concentrations and for different durations. In all conditions that received FGF2, more caudal MN identities were induced without the need of delaying RA addition (FIG. 4A-D). Then, the extent of caudalization varied with FGF parameters. Exposing progenitors from D3 to D9 to increasing concentrations of FGF2 induced progressively more caudal MN subtypes. Caudal brachial HOXC8 MNs were induced at 15 ng/ml (68.2%, 8.6-fold increase) and HOXC9+/HOXC6-thoracic MNs at 60 ng/ml (58.9%, 82.5-fold increase) while 120 ng/ml reduced slightly more HOXC6 MNs (FIGS. 4A-D, S6A-H). Similar results were obtained with FGF8 (FIG. S3B). FGFs acted directly and rapidly on axial progenitors as addition of 120 ng/ml FGF2 for 24 h or 48 h induced MNs of a caudal brachial or mid-thoracic identity (49.7% HOXC6, 79.4% HOXC8, 14.7% HOXC9, 47.9% FOXP1^{high}/SCIP for 24 h) (7.5% HOXC6, 76.4% HOXC9, 13.2% FOXP1^{high}/SCIP for 48 h) (FIGS. 4A-C, S3C-H).

To determine whether this caudalizing effect was underlined by an accelerated induction of brachial and thoracic HOX genes we performed real-time PCR analysis 24 h and 48 h post FGF2 treatment. We observed a precocious increase in HOXC8, HOXC9, HOXD9 and HOXC10 expression compared to RA/SAG controls (FIG. 4F).

Hence, a precocious increase in FGF signaling accelerates the induction of caudal HOX genes in early axial progenitors resulting in the specification of more caudal cell types within the same differentiation timeline (14 days). These results demonstrate that the levels or duration of FGF signaling dynamically pace the tempo of HOX collinear activation during human axial progenitor differentiation.

FGF and GDF11 Synergize to Further Accelerate the HOX Clock

FGF2 accelerated HOX induction to generate MNs up to the mid-thoracic level suggesting that early axial progenitors might not be competent to generate more caudal segments. Alternatively, other extrinsic factors might be required to further accelerate the induction of HOX genes and promote more caudal identities. GDF11 is a member of the TGF□ family implicated in the control of axial elongation, MN subtype specification and is required for the expression of the most 5′ HOX gene starting from the 10 paralogs in vivo and in vitro in late NMP-like cells (Aires et al., 2019; Gaunt et al., 2013; Lippmann et al., 2015; Liu, 2006; Liu et al., 2001; McPherron et al., 1999; Peljto et al., 2010). We exposed D3 early progenitors to a combination of RA and GDF11 (25 ng/ml) for 24 h or 48 h. 24 h GDF11 induced caudal brachial and anterior thoracic MNs (58.7% HOXC8, 41.0% HOXC9); the latest category increasing after 48 h (FIGS. 4A, E, S4A). However, as with FGF2, none of the more caudal identities were observed. In chick, exposure of spinal cord explants to combination of FGF2 and GDF11 promoted more caudal MNs than the two factors separately (Liu et al., 2001). We thus tested whether this combination might accelerate the clock and promote caudal thoracic or lumbar identities. 24 h after FGF2/GDF11 treatment, HOXC9, HOXD9 and HOXC10 mRNAs were strongly induced (respectively 65.4, 2774.77 and 3329.44-fold compared to controls) and further increased after 48 h (FIG. 4F). In agreement, a 24 h (D3-D4) treatment induced, on day 14, caudal thoracic MNs (88.2% HOXC9 and 46.7% HOXD9) and lumbar HOXC10 MNs after 48 h of exposure (11.1%) (FIGS. 4B, E, S4B-E).

Hence, axial progenitors are competent to produce the different spinal rostro-caudal identities from brachial to lumbar. Changes in parameters of exposure to FGFs and GDF1 1 (concentrations, durations and combinations) dynamically pace the speed at which the HOX clock proceed resulting in the specification of early or later born MN subtypes within the same timeline of differentiation.

Table S1—Transcriptomic Data and List of Markers Defining Axial Progenitors and Other Developmentally Related Cell Types

The first panel displays the normalized number of reads for each gene for hESC, D2, D3 and D4 replicates. The following panels display the list of genes enriched (FC≥2.0, p-value<0.05) in the indicated comparison. The last panel displays the list of genes that identify specific cell types (allantois, mesodermal progenitors) and the references used to construct these lists.

TABLE S2
Growth factors and small molecules.

| Molecule | Concentration | | Supplier | Reference |
|---|---|---|---|---|
| Y-27632 (2HCl) | 10 | μM | Stemgent | 04-0012-H-10; |
| | | | | CAS: 146986-50-7 |
| Y-27632 (2HCl) | 10 | μM | Stem Cell | 72304; |
| | | | Technologies | CAS: 129830-38-2 |
| RA | 100 | nM | Sigma-Aldrich | R2625; CAS: |
| | | | | 302-79-4 |
| LDN193189 | 100 | nM | Stemgent | STE04-0074; |
| | | | | CAS: 1062368-24-4 |
| SB431542 | 20 | μM | Stemgent | STE04-0010-10; |
| | | | | CAS: 301836-41-9 |
| CHIR99021 | 3 or 4 | μM | Selleckchem | S1263; CAS: |
| (CT99021) | | | | 252917-06-9 |
| SAG | 500 | nM | Merk | 566660; CAS: |
| | | | | 364590-63-6 |
| SCH772984 | 100 | nM | Selleckchem | S7101; CAS: |
| | | | | 942183-80-4 |
| PD173074 | 100 | nM | Selleckchem | S1264; CAS: |
| | | | | 219580-11-7 |
| PD0325901 | 5 | μM | Selleckchem | S1036; CAS: |
| | | | | 391210-10-9 |
| Recombinant | 15-120 | ng/ml | Peprotech | 100-18B, |
| Human FGF | | | | accession Number: |
| basic (FGF2) | | | | P09038 |
| Recombinant | 120 | ng/ml | Peprotech | 100-25; accession |
| Human/Murine | | | | Number: P55075 |
| FGF-8b | | | | |
| Recombinant | 10-50 | ng/ml | Peprotech | 120-11; Accession |
| Human/Murine/ | | | | number: O95390 |
| Rat GDF-11 | | | | |
| DAPT | 10 | μM | Stemgent | STE04-0041 |
| | | | | CAS: 208255-80-5 |

TABLE S3
Antibodies.

| | HOST | | | |
|---|---|---|---|---|
| Target | species | Dilution | References | Provider |
| CDX2 | Rabbit | 1:1000 | AB76541 | Abcam |
| | | | RRID: AB_1523334 | |
| FOXP1 | Rabbit | 1:32000 | AB16645 | Abcam |
| | | | RRID: AB_732428 | |
| FOXP1 | Mouse | 1:3000 | MAB45341 | R&D systems |
| | | | RRID: AB_2107101 | |
| HB9 | Mouse | 1:50 | 81.5C10 | DSHB |
| | | | RRID: AB_2145209 | |
| HOXA5 | Rabbit | 1:1000 | ARP31447_P050 | Sigma-Aldrich |
| | | | RRID: AB_2046241 | |
| HOXA5 | Guinea pig | 1:1000 | Cu570 | H. Wichterle |
| HOXC5 | Rabbit | 1:1000 | HPA026794 | Sigma-Aldrich |
| | | | RRID: AB_10612118 | |
| HOXC6 | Rabbit | 1:8000 | ARP38484_P50 | Euromedex |
| | | | RRID: AB_10866814 | |
| HOXC8 | Mouse | 1:10000 | BLE920502 | Biolegend |
| HOXC9 | Guinea pig | 1:300 | — | J. Dasen |
| HOXD9 | Rabbit | 1:1500 | SC-8320 | Santa-Cruz |
| HOXC10 | Rabbit | 1:800 | HPA053919 | Sigma-Aldrich |
| | | | RRID: AB_2682308 | |
| HuC/D | Rabbit | 1:500 | A21271 | Invitrogen |
| | | | RRID: AB_221448 | |
| ISL1 | Goat | 1:1000 | GT15051 | Neuromics |
| | | | RRID: AB_2126323 | |
| NEUROFILAMENT | Chicken | 1:500 | CH22105 | Neuromics |
| | | | RRID: AB_2737102 | |
| SCIP | Rabbit | 1:1000 | AB126746 | Abcam |
| | | | RRID: AB_11130256 | |
| SOX2 | Mouse | 1:200 | AB79351 | Abcam |
| | | | RRID: AB_10710406 | |
| TBXT/BRACHURY | Goat | 1:800 | AF2085 | R&D systems |
| | | | RRID: AB_2200235 | |

Tested antibodies with no staining or unspecific one

| | | | |
|---|---|---|---|
| HOXC8 | Mouse | sc-517007 | Santa Cruz |
| HOXC9 | Rabbit | ARP35813 (Middle | Aviva Sytems |
| | | region) | Biology |
| | | RRID: AB_938161 | |
| HOXC9 | Rabbit | H2041 n-terminal | Sigma-Aldrich |
| | | region | |
| | | RRID: AB_10603946 | |
| HOXC9 | Rabbit | H1916 | Sigma-Aldrich |
| | | RRID: AB_10604162 | |
| HOXC10 | Mouse | 5G12-10 supernatant | DSHB |
| | | RRID: AB_528286 | |

TABLE S4
Real time PCR Primers.

| Primer Sequences | Targeted Gene |
|---|---|
| CCCACCGTGTTCTTCGACAT | CYCLOA |
| CCAGTGCTCAGAGCACGAAA | CYCLOA |
| GGAGATCATAGTTCCGTGAGC | HOXA5 |
| GCTGAGATCCATGCCATTGT | HOXA5 |
| CCGCCACAGATTTACCCGT | HOXC5 |
| AGTCTGGTAGCGCGTGTAAC | HOXC5 |
| CCAGGACCAGAAAGCCAGTA | HOXC6 |
| GTTAGGTAGCGATTGAAGTGAAA | HOXC6 |
| CCTCCGCCAACACTAACAGT | HOXC8 |
| CAAGGTCTGATACCGGCTGT | HOXC8 |
| ACCTCCTAGCGTCCAGGTTT | HOXC9 |
| CGAAGCTACAGGACGGAAA | HOXC9 |
| TCGCTGAAGGAGGAGGAGA | HOXD9 |
| CAAACACCCACAAAGGAAAAC | HOXD9 |
| ACCTCGGATAACGAAGCGAAA | HOXC10 |
| TCCAGCGTCTGGTGTTTAGT | HOXC10 |
| GAACCTGTGCGAGTGGATG | CDX2 |
| GGATGGTGATGTAGCGACTG | CDX2 |
| TCTTTGCTTGGGAAATCCG | PAX6 |
| CTGCCCGTTCAACATCCTTAG | PAX6 |
| GATGCACAACTCGGAGATCA | SOX1 |
| GTCCTTCTTGAGCAGCGTCT | SOX1 |

### DISCUSSION

Overall, we report the selective generation of axial progenitors from hPSCs demonstrated by combination of specific markers, detailed transcriptomic analysis and their ability to generate cell types found at distinct rostro-caudal levels. Importantly, we observed line to line differences in the optimal concentration of CHIR necessary to potently induce an axial progenitor state for the subsequent reliable induction of caudal cell types. With efficient access to axial progenitors we then show that changes in the concentration, duration and combination of the caudalizing factors FGFs and GDF1 1 control the speed at which the temporal collinear activation of HOX genes occurs. The pace of the HOX clock is thus dynamically encoded by the parameters of exposure to extrinsic cues. The sequential changes in chromatin structure occurring along HOX complexes during their activation do not appear to act as an intrinsic timer actuated and stopped by extrinsic factors (Bel-Vialar et al., 2002; Del Corral and Storey, 2004; Kimelman and Martin, 2012; Lippmann et al., 2015; Mazzoni et al., 2013; Narendra et al., 2015; Noordermeer et al., 2011; Noordermeer et al., 2014; Soshnikova and Duboule, 2009; Tschopp et al., 2009). Our results might provide a molecular basis for the observation that heterochronic grafting of “old” axial progenitors to a “younger” caudal stem zone reverted their HOX profile to the “young” one (McGrew et al., 2008; Wymeersch et al., 2019). The pacing of the HOX clock by secreted factors might ensure a community effect to synchronize HOX expression between neighboring progenitors which could allow the emergence of expression domains at tissue level (Durston, 2019). Furthermore, FGFs and GDFs also control the maintenance of the stem cell pool and axial elongation (Aires et al., 2019; Boulet and Capecchi, 2012; Jurberg et al., 2013; Mallo et al., 2009; McPherron et al., 1999). A common mechanism to control rostro-caudal extension of the body axis together with Hox gene induction would be a parsimonious way to couple morphogenesis and patterning (Denans et al., 2015; Young et al., 2009). In a bioengineering perspective, HOX clock extrinsic control provides a simple mean to engineer cell types of defined “rostro-caudal” identities from PSCs. It shortens temporal requirements for the generation of cells born at different time of development during axial elongation. This allows the synchronous engineering with an unprecedented efficiency and precision of human MN subtypes with defined rostro-caudal identities. In particular, we provide the first evidence for the generation of putative hand-controlling MNs (Mendelsohn et al., 2017). MN subtypes display differential vulnerabilities in disease and in spinal injuries. Our results will provide access to a long awaited resource for the modeling of these incurable diseases and more controlled source of cells for putative cell therapy approaches. (Abati et al., 2019; An et al., 2019; Baloh et al., 2018; Nijssen et al., 2017; Ragagnin et al., 2019; Sances et al., 2016; Steinbeck and Studer, 2015; Tung et al., 2019). As HOX play a central role in instructing cell diversification in the three lineages, controlled manipulation of the HOX clock has a great potential for cell engineering besides MN subtypes. Our strategy is likely expandable to other axial stem cell derivatives such as paraxial mesoderm that generate the different muscles of the body with clear applications for the modeling of neuromuscular diseases (Bakooshli et al., 2019; Diaz-Cuadros et al., 2020; Faustino Martins et al., 2020; Frith et al., 2018; Machado et al., 2019; Matsuda et al., 2020; Osaki et al., 2020; Pourquié et al., 2018; Steinbeck et al., 2015; Verrier et al., 2018). More broadly, the temporal generation of distinct types of neurons or glia from the same progenitor domain is a widely used strategy to increase cell diversity in the nervous system (Dias et al., 2014; Kohwi and Doe, 2013; Oberst et al., 2019a; Rossi et al., 2017). Extrinsic cues play important roles in the unfolding of these temporal sequences (Kawaguchi, 2019; Oberst et al., 2019a; Oberst et al., 2019b; Syed et al., 2017; Tiberi et al., 2012). Extrinsic manipulation of the temporality of these lineages should improve the generation of early and late-born cells for both basic research, disease modeling and cell therapy.

### REFERENCES

Abati, E., Bresolin, N., Comi, G. and Corti, S. (2019). Advances, Challenges, and Perspectives in Translational Stem Cell Therapy for Amyotrophic Lateral Sclerosis. Mol. Neurobiol. 56, 6703-6715.
Aires, R., de Lemos, L., Nóvoa, A., Jurberg, A. D., Mascrez, B., Duboule, D. and Mallo, M. (2019). Tail Bud Progenitor Activity Relies on a Network Comprising Gdf11, Lin28, and Hox13 Genes. Dev. Cell 48, 383-395.e8.
Amoroso, M. W., Croft, G. F., Williams, D. J., O'Keeffe, S., Carrasco, M. A., Davis, A. R., Roybon, L., Oakley, D. H., Maniatis, T., Henderson, C. E., et al. (2013). Accelerated high-yield generation of limb-innervating motor neurons from human stem cells. J. Neurosci. 33, 574-586.
An, D., Fujiki, R., Iannitelli, D. E., Smerdon, J. W., Maity, S., Rose, M. F., Gelber, A., Wanaselja, E. K., Yagudayeva, I., Lee, J. Y., et al. (2019). Stem cell-derived cranial and spinal motor neurons reveal proteostatic differences between ALS resistant and sensitive motor neurons. Elife 8.
Attardi, A., Fulton, T., Florescu, M., Shah, G., Muresan, L., Lenz, M. O., Lancaster, C., Huisken, J., van Oudenaarden, A. and Steventon, B. (2018). Neuromesodermal progenitors are a conserved source of spinal cord with divergent growth dynamics. Development 145, dev166728.
Bakooshli, M. A., Lippmann, E. S., Mulcahy, B., Iyer, N., Nguyen, C. T., Tung, K., Stewart, B. A., Van Den Dorpel, H., Fuehrmann, T., Shoichet, M., et al. (2019). A 3d culture model of innervated human skeletal muscle enables studies of the adult neuromuscular junction. Elife 8.
Baloh, R. H., Glass, J. D. and Svendsen, C. N. (2018). Stem cell transplantation for amyotrophic lateral sclerosis. Curr. Opin. Neurol. 31, 655-661.
Bel-Vialar, S., Itasaki, N. and Krumlauf, R. (2002). Initiating Hox gene expression: In the early chick neural tube differential sensitivity to FGF and RA signaling subdivides the HoxB genes in two distinct groups. Development 129, 5103-5115.
Bell, S. W., Brown, M. J. C. and Hems, T. J. (2017). Refinement of myotome values in the upper limb: Evidence from brachial plexus injuries. Surgeon 15, 1-6.
Bialecka, M., Wilson, V. and Deschamps, J. (2010). Cdx mutant axial progenitor cells are rescued by grafting to a wild type environment. Dev. Biol. 347, 228-234.
Boulet, A. M. and Capecchi, M. R. (2012). Signaling by FGF4 and FGF8 is required for axial elongation of the mouse embryo. Dev. Biol. 371, 235-245.
Briscoe, J. and Novitch, B. G. (2008). Regulatory pathways linking progenitor patterning, cell fates and neurogenesis in the ventral neural tube. Philos. Trans. R. Soc. B Biol. Sci. 363, 57-70.
Cambray, N. and Wilson, V. (2007). Two distinct sources for a population of maturing axial progenitors. Development 134, 2829-2840.
Carpenter, A. E., Jones, T. R., Lamprecht, M. R., Clarke, C., Kang, I. H., Friman, O., Guertin, D. A., Chang, J. H., Lindquist, R. A., Moffat, J., et al. (2006). CellProfiler: Image analysis software for identifying and quantifying cell phenotypes. Genome Biol.
Catela, C., Shin, M. M., Lee, D. H., Liu, J. P. and Dasen, J. S. (2016). Hox Proteins Coordinate Motor Neuron Differentiation and Connectivity Programs through Ret/Gfra Genes. Cell Rep. 14, 1901-1915.
Dasen, J. S. (2017). Master or servant? emerging roles for motor neuron subtypes in the construction and evolution of locomotor circuits. Curr. Opin. Neurobiol. 42, 25-32.
Dasen, J. S., Liu, J. P. and Jessell, T. M. (2003). Motor neuron columnar fate imposed by sequential phases of Hox-c activity. Nature 425, 926-933.
Dasen, J. S., De Camilli, A., Wang, B., Tucker, P. W. and Jessell, T. M. (2008). Hox Repertoires for Motor Neuron Diversity and Connectivity Gated by a Single Accessory Factor, FoxP1. Cell 134, 304-316.
Del Corral, R. D. and Storey, K. G. (2004). Opposing FGF and retinoid pathways: A signalling switch that controls differentiation and patterning onset in the extending vertebrate body axis. BioEssays 26, 857-869.
Denans, N., limura, T. and Pourquié, O. (2015). Hox genes control vertebrate body elongation by collinear Wnt repression. Elife 2015.
Denham, M., Hasegawa, K., Menheniott, T., Rollo, B., Zhang, D., Hough, S., Alshawaf, A., Febbraro, F., Ighaniyan, S., Leung, J., et al. (2015). Multipotent caudal neural progenitors derived from human pluripotent stem cells that give rise to lineages of the central and peripheral nervous system. Stem Cells 33, 1759-1770.
Deschamps, J. and Duboule, D. (2017). Embryonic timing, axial stem cells, chromatin dynamics, and the Hox clock. Genes Dev. 31, 1406-1416.
Dias, J. M., Alekseenko, Z., Applequist, J. M. and Ericson, J. (2014). Tgfβ signaling regulates temporal neurogenesis and potency of neural stem cells in the CNS. Neuron 84, 927-939.
Diaz-Cuadros, M., Wagner, D. E., Budjan, C., Hubaud, A., Tarazona, O. A., Donelly, S., Michaut, A., Al Tanoury, Z., Yoshioka-Kobayashi, K., Niino, Y., et al. (2020). In vitro characterization of the human segmentation clock. Nature 580, 113-118.
Du, Z.-W., Chen, H., Liu, H., Lu, J., Qian, K., Huang, C.-L., Zhong, X., Fan, F. and Zhang, S.-C. (2015). Generation and expansion of highly pure motor neuron progenitors from human pluripotent stem cells. Nat. Commun. 6, 6626.
Durston, A. J. (2019). Some Questions and Answers About the Role of Hox Temporal Collinearity in Vertebrate Axial Patterning. Front. Cell Dev. Biol. 7.
Duval, N., Vaslin, C., Barata, T. C., Frarma, Y., Contremoulins, V., Baudin, X., Nedelec, S. and Ribes, V. C. (2019). Bmp4 patterns smad activity and generates stereotyped cell fate organization in spinal organoids. Dev. 146, 24-33.
Ebisuya, M. and Briscoe, J. (2018). What does time mean in development? Development 145, dev164368.
Edri, S., Hayward, P., Jawaid, W. and Arias, A. M. (2019). Neuro-mesodermal progenitors (NMPs): A comparative study between pluripotent stem cells and embryo-derived populations. Dev. 146.
Evtouchenko, L., Studer, L., Spencer, C., Dreher, E. and Seiler, R. W. (1996). A mathematical model for the estimation of human embryonic and fetal age. Cell Transplant. 5, 453-464.
Faustino Martins, J. M., Fischer, C., Urzi, A., Vidal, R., Kunz, S., Ruffault, P. L., Kabuss, L., Hube, I., Gazzerro, E., Birchmeier, C., et al. (2020). Self-Organizing 3D Human Trunk Neuromuscular Organoids. Cell Stem Cell 26, 172-186.e6.
Frith, T. J. R., Granata, I., Wind, M., Stout, E., Thompson, O., Neumann, K., Stavish, D., Heath, P. R., Ortmann, D., Hackland, J. O. S., et al. (2018). Human axial progenitors generate trunk neural crest cells in vitro. Elife 7.
Gaunt, S. J. (1991). Expression patterns of mouse hox genes: Clues to an understanding of developmental and evolutionary strategies. BioEssays 13, 505-513.
Gaunt, S. J., George, M. and Paul, Y. L. (2013). Direct activation of a mouse Hoxd11 axial expression enhancer by Gdf1 1/Smad signalling. Dev. Biol. 383, 52-60.
Gouti, M., Tsakiridis, A., Wymeersch, F. J., Huang, Y., Kleinjung, J., Wilson, V. and Briscoe, J. (2014). In vitro generation of neuromesodermal progenitors reveals distinct roles for wnt signalling in the specification of spinal cord and paraxial mesoderm identity. PLoS Biol. 12, e1001937.
Gouti, M., Delile, J., Stamataki, D., Wymeersch, F. J., Huang, Y., Kleinjung, J., Wilson, V. and Briscoe, J. (2017). A Gene Regulatory Network Balances Neural and Mesoderm Specification during Vertebrate Trunk Development. Dev. Cell 41, 243-261.e7.
Henrique, D., Abranches, E., Verrier, L. and Storey, K. G. (2015). Neuromesodermal progenitors and the making of the spinal cord. Dev. 142, 2864-2875.
Izpisda-Belmonte, J. C., Falkenstein, H., Dollé, P., Renucci, A. and Duboule, D. (1991). Murine genes related to the Drosophila AbdB homeotic genes are sequentially expressed during development of the posterior part of the body. EMBO J. 10, 2279-2289.
Jung, H., Lacombe, J., Mazzoni, E. O., Liem, K. F., Grinstein, J., Mahony, S., Mukhopadhyay, D., Gifford, D. K., Young, R. A., Anderson, K. V., et al. (2010). Global Control of Motor Neuron Topography Mediated by the Repressive Actions of a Single Hox Gene. Neuron 67, 781-796.
Jurberg, A. D., Aires, R., Varela-Lasheras, I., Nóvoa, A. and Mallo, M. (2013). Switching axial progenitors from producing trunk to tail tissues in vertebrate embryos. Dev. Cell 25, 451-462.
Kawaguchi, A. (2019). Temporal patterning of neocortical progenitor cells: How do they know the right time? Neurosci. Res. 138, 3-11.
Kimelman, D. and Martin, B. L. (2012). Anterior-posterior patterning in early development: Three strategies. Wiley Interdiscip. Rev. Dev. Biol. 1, 253-266.
Koch, F., Scholze, M., Wittler, L., Schifferl, D., Sudheer, S., Grote, P., Timmermann, B., Macura, K. and Herrmann, B. G. (2017). Antagonistic Activities of Sox2 and Brachyury Control the Fate Choice of Neuro-Mesodermal Progenitors. Dev. Cell 42, 514-526.e7.
Kohwi, M. and Doe, C. Q. (2013). Temporal fate specification and neural progenitor competence during development. Nat. Rev. Neurosci. 14, 823-838.
Lambrot, R., Coffigny, H., Pairault, C., Donnadieu, A. C., Frydman, R., Habert, R. and Rouiller-Fabre, V. (2006). Use of organ culture to study the human fetal testis development: Effect of retinoic acid. J. Clin. Endocrinol. Metab. 91, 2696-2703.
Li, X. J., Du, Z. W., Zarnowska, E. D., Pankratz, M., Hansen, L. O., Pearce, R. A. and Zhang, S. C. (2005). Specification of motoneurons from human embryonic stem cells. Nat. Biotechnol. 23, 215-221.
Lippmann, E. S., E. williams, C., Ruhl, D. A., Estevez-Silva, M. C., Chapman, E. R., Coon, J. J. and Ashton, R. S. (2015). Deterministic HOX patterning in human pluripotent stem cell-derived neuroectoderm. Stem Cell Reports 4, 632-644.
Liu, J. P. (2006). The function of growth/differentiation factor 11 (Gdf11) in rostrocaudal patterning of the developing spinal cord. Development 133, 2865-2874.
Liu, J. P., Laufer, E. and Jessell, T. M. (2001). Assigning the positional identity of spinal motor neurons: Rostrocaudal patterning of Hox-c expression by FGFs, Gdf11, and retinoids. Neuron 32, 997-1012.
Lunn, J. S., Fishwick, K. J., Halley, P. A. and Storey, K. G. (2007). A spatial and temporal map of FGF/Erk1/2 activity and response repertoires in the early chick embryo. Dev. Biol. 302, 536-552.
Machado, C. B., Kanning, K. C., Kreis, P., Stevenson, D., Crossley, M., Nowak, M., Iacovino, M., Kyba, M., Chambers, D., Blanc, E., et al. (2014). Reconstruction of phrenic neuron identity in embryonic stem cell-derived motor neurons. Dev. 141, 784-794.
Machado, C. B., Pluchon, P., Harley, P., Rigby, M., Sabater, V. G., Stevenson, D. C., Hynes, S., Lowe, A., Burrone, J., Viasnoff, V., et al. (2019). In Vitro Modeling of Nerve-Muscle Connectivity in a Compartmentalized Tissue Culture Device. Adv. Biosyst.
Mallo, M., Vinagre, T. and Carapugo, M. (2009). The road to the vertebral formula. Int. J. Dev. Biol. 53, 1469-1481.
Matsuda, M., Yamanaka, Y., Uemura, M., Osawa, M., Saito, M. K., Nagahashi, A., Nishio, M., Guo, L., Ikegawa, S., Sakurai, S., et al. (2020). Recapitulating the human segmentation clock with pluripotent stem cells. Nature 580, 124-129.
Maury, Y., Côme, J., Piskorowski, R. A., Salah-Mohellibi, N., Chevaleyre, V., Peschanski, M., Martinat, C. and Nedelec, S. (2015). Combinatorial analysis of developmental cues efficiently converts human pluripotent stem cells into multiple neuronal subtypes. Nat. Biotechnol. 33, 89-96.
Mazzoni, E. O., Mahony, S., Peljto, M., Patel, T., Thornton, S. R., McCuine, S., Reeder, C., Boyer, L. A., Young, R. A., Gifford, D. K., et al. (2013). Saltatory remodeling of Hox chromatin in response to rostrocaudal patterning signals. Nat. Neurosci. 16, 1191-1198.
McGrew, M. J., Sherman, A., Lillico, S. G., Ellard, F. M., Radcliffe, P. A., Gilhooley, H. J., Mitrophanous, K. A., Cambray, N., Wilson, V. and Sang, H. (2008). Localised axial progenitor cell populations in the avian tail bud are not committed to a posterior Hox identity. Development 135, 2289-2299.
McPherron, A. C., Lawler, A. M. and Lee, S. J. (1999). Regulation of anterior/posterior patterning of the axial skeleton by growth/differentiation factor 11. Nat. Genet. 22, 260-264.
Mendelsohn, A. I., Dasen, J. S. and Jessell, T. M. (2017). Divergent Hox Coding and Evasion of Retinoid Signaling Specifies Motor Neurons Innervating Digit Muscles. Neuron 93, 792-805.e4.
Metzis, V., Steinhauser, S., Pakanavicius, E., Gouti, M., Stamataki, D., Ivanovitch, K., Watson, T., Rayon, T., Mousavy Gharavy, S. N., Lovell-Badge, R., et al. (2018). Nervous System Regionalization Entails Axial Allocation before Neural Differentiation. Cell 175, 1105-1118.e17.
Narendra, V., Rocha, P. P., An, D., Raviram, R., Skok, J. A., Mazzoni, E. O. and Reinberg, D. (2015). CTCF establishes discrete functional chromatin domains at the Hox clusters during differentiation. Science (80-.). 347, 1017-1021.
Neijts, R., Amin, S., van Rooijen, C. and Deschamps, J. (2017). Cdx is crucial for the timing mechanism driving colinear Hox activation and defines a trunk segment in the Hox cluster topology. Dev. Biol. 422, 146-154.
Nijssen, J., Comley, L. H. and Hedlund, E. (2017). Motor neuron vulnerability and resistance in amyotrophic lateral sclerosis. Acta Neuropathol. 133, 863-885.
Noordermeer, D., Leleu, M., Splinter, E., Rougemont, J., De Laat, W. and Duboule, D. (2011). The dynamic architecture of Hox gene clusters. Science (80-.). 334, 222-225.
Noordermeer, D., Leleu, M., Schorderet, P., Joye, E., Chabaud, F. and Duboule, D. (2014). Temporal dynamics and developmental memory of 3D chromatin architecture at Hox gene loci. Elife 2014.
Oberst, P., FiBvre, S., Baumann, N., Concetti, C., Bartolini, G. and Jabaudon, D. (2019a). Temporal plasticity of apical progenitors in the developing mouse neocortex. Nature 573, 370-374.
Oberst, P., Agirman, G. and Jabaudon, D. (2019b). Principles of progenitor temporal patterning in the developing invertebrate and vertebrate nervous system. Curr. Opin. Neurobiol. 56, 185-193.
Ogura, T., Sakaguchi, H., Miyamoto, S. and Takahashi, J. (2018). Three-dimensional induction of dorsal, intermediate and ventral spinal cord tissues from human pluripotent stem cells. Development 145, dev162214.
Olivera-Martinez, I., Harada, H., Halley, P. A. and Storey, K. G. (2012). Loss of FGF-Dependent Mesoderm Identity and Rise of Endogenous Retinoid Signalling Determine Cessation of Body Axis Elongation. PLoS Biol. 10.
Osaki, T., Uzel, S. G. M. and Kamm, R. D. (2020). On-chip 3D neuromuscular model for drug screening and precision medicine in neuromuscular disease. Nat. Protoc. 15, 421-449.
Patani, R., Hollins, A. J., Wishart, T. M., Puddifoot, C. A., Alvarez, S., de Lera, A. R., Wyllie, D. J., Compston, D. A., Pedersen, R. A., Gillingwater, T. H., Hardingham, G. E., Allen, N. D., & Chandran, S. (2011). Retinoid-independent motor neurogenesis from human embryonic stem cells reveals a medial columnar ground state. Nature communications, 2, 214. https://doi.org/10.1038/ncomms1216
Peljto, M., Dasen, J. S., Mazzoni, E. O., Jessell, T. M. and Wichterle, H. (2010). Functional diversity of ESC-derived motor neuron subtypes revealed through intraspinal transplantation. Cell Stem Cell 7, 355-366.
Philippidou, P. and Dasen, J. S. (2013). Hox Genes: Choreographers in Neural Development, Architects of Circuit Organization. Neuron 80, 12-34.
Philippidou, P., Walsh, C. M., Aubin, J., Jeannotte, L. and Dasen, J. S. (2012). Sustained Hox5 gene activity is required for respiratory motor neuron development. Nat. Neurosci. 15, 1636-1644.
Pourquié, O., Al Tanoury, Z. and Chal, J. (2018). The Long Road to Making Muscle In Vitro. In Current Topics in Developmental Biology, pp. 123-142.
Ragagnin, A. M. G., Shadfar, S., Vidal, M., Jamali, M. S. and Atkin, J. D. (2019). Motor neuron susceptibility in ALS/FTD. Front. Neurosci. 13.
Ribes, V., Le Roux, I., Rhinn, M., Schuhbaur, B. and Dolle, P. (2009). Early mouse caudal development relies on crosstalk between retinoic acid, Shh and Fgf signalling pathways. Development 136, 665-676.
Rossi, A. M., Fernandes, V. M. and Desplan, C. (2017). Timing temporal transitions during brain development. Curr. Opin. Neurobiol. 42, 84-92.
Rousso, D. L., Gaber, Z. B., Wellik, D., Morrisey, E. E. and Novitch, B. G. (2008). Coordinated Actions of the Forkhead Protein Foxpl and Hox Proteins in the Columnar Organization of Spinal Motor Neurons. Neuron 59, 226-240.
Routal, R. V. and Pal, G. P. (1999). A study of motoneuron groups and motor columns of the human spinal cord. J. Anat. 195, 211-224.
Sances, S., Bruijn, L. I., Chandran, S., Eggan, K., Ho, R., Klim, J. R., Livesey, M. R., Lowry, E., Macklis, J. D., Rushton, D., et al. (2016). Modeling ALS with motor neurons derived from human induced pluripotent stem cells. Nat. Neurosci. 19, 542-553.
Schindelin, J., Arganda-Carreras, I., Frise, E., Kaynig, V., Longair, M., Pietzsch, T., Preibisch, S., Rueden, C., Saalfeld, S., Schmid, B., et al. (2012). Fiji: An open-source platform for biological-image analysis. Nat. Methods 9, 676-682.
Soshnikova, N. and Duboule, D. (2009). Epigenetic temporal control of mouse hox genes in vivo. Science (80-.). 324, 1321-1323.
Steinbeck, J. A. and Studer, L. (2015). Moving stem cells to the clinic: Potential and limitations for brain repair. Neuron 86, 187-206.
Steinbeck, J. A., Choi, S. J., Mrejeru, A., Ganat, Y., Deisseroth, K., Sulzer, D., Mosharov, E. V. and Studer, L. (2015). Optogenetics enables functional analysis of human embryonic stem cell-derived grafts in a Parkinson's disease model. Nat. Biotechnol. 33, 204-209.
Syed, M. H., Mark, B. and Doe, C. Q. (2017). Playing Well with Others: Extrinsic Cues Regulate Neural Progenitor Temporal Identity to Generate Neuronal Diversity. Trends Genet. 33, 933-942.
Tiberi, L., Vanderhaeghen, P. and van den Ameele, J. (2012). Cortical neurogenesis and morphogens: Diversity of cues, sources and functions. Curr. Opin. Cell Biol. 24, 269-276.
Tschopp, P., Tarchini, B., Spitz, F., Zakany, J. and Duboule, D. (2009). Uncoupling time and space in the collinear regulation of Hox genes. PLoS Genet. 5.
Tung, Y. T., Peng, K. C., Chen, Y. C., Yen, Y. P., Chang, M., Thams, S. and Chen, J. A. (2019). Mir-17-92 Confers Motor Neuron Subtype Differential Resistance to ALS-Associated Degeneration. Cell Stem Cell 25, 193-209.e7.
Tzouanacou, E., Wegener, A., Wymeersch, F. J., Wilson, V. and Nicolas, J. F. (2009). Redefining the Progression of Lineage Segregations during Mammalian Embryogenesis by Clonal Analysis. Dev. Cell 17, 365-376.
Verrier, L., Davidson, L., Gierlinśki, M., Dady, A. and Storey, K. G. (2018). Neural differentiation, selection and transcriptomic profiling of human neuromesodermal progenitor-like cells in vitro. Dev. 145, dev166215.
Wang, J., Vasaikar, S., Shi, Z., Greer, M. and Zhang, B. (2017). WebGestalt 2017: A more comprehensive, powerful, flexible and interactive gene set enrichment analysis toolkit. Nucleic Acids Res. 45, W130-W137.
Wymeersch, F. J., Huang, Y., Blin, G., Cambray, N., Wilkie, R., Wong, F. C. K. and Wilson, V. (2016). Position-dependent plasticity of distinct progenitor types in the primitive streak. Elife 5.
Wymeersch, F. J., Skylaki, S., Huang, Y., Watson, J. A., Economou, C., Marek-Johnston, C., Tomlinson, S. R. and Wilson, V. (2019). Transcriptionally dynamic progenitor populations organised around a stable niche drive axial patterning. Dev. 146, dev168161.
Young, T., Rowland, J. E., van de Ven, C., Bialecka, M., Novoa, A., Carapuco, M., van Nes, J., de Graaff, W., Duluc, I., Freund, J. N., et al. (2009). Cdx and Hox Genes Differentially Regulate Posterior Axial Growth in Mammalian Embryos. Dev. Cell.

1-13. (canceled)

14. An in vitro, or an ex vivo method for controlling rostro-caudal identity of human neuron subpopulations comprising culturing axial progenitors in a culture medium comprising retinoic acid (RA), a Hedgehog signalling pathway agonist, and optionally a fibroblast growth factor receptor (FGFR) agonist or a transforming growth factor (TGF)/activin/nodal signalling pathway activator, thereby obtaining spinal motor neuron progenitors, wherein more and more caudal motor neurons identities are obtained by delayed addition of the retinoic acid in the culture medium, or by addition of the retinoic acid in combination with the FGFR agonist or the TGF/activin/nodal signalling pathway activator.

15. The method according to claim 14 further comprising:
exposing human pluripotent stem cells (hPSCs) in a culture medium comprising a Wnt signalling pathway activator to obtain the axial progenitors, and
exposing the axial progenitors to the RA and the Hedgehog signalling pathway agonist, optionally combined with the FGFR agonist or the TGF/activin/nodal signalling pathway activator in the culture medium to obtain spinal motor neuron progenitors,
wherein more and more caudal motor neuron identities are generated either by delaying the exposure of the axial progenitors to the RA with regard to the exposure to the Wnt signalling pathway activator or by exposing the axial progenitors to the RA in combination with an increased concentration of or an increased duration of exposure to the FGFR agonist or the TGF/activin/nodal signalling pathway activator.

16. The method according to claim 14 further comprising:
exposing the spinal motor neuron progenitors to a notch pathway inhibitor in the culture medium.

17. The method of claim 15, wherein the human pluripotent stem cells (hPSCs) are exposed to the Wnt signalling pathway activator in combination with a Bone Morphogenetic Protein (BMP) signalling pathway inhibitor, and the TGF/activin/nodal signalling pathway inhibitor.

18. The method of claim 17, wherein:
the BMP signalling pathway inhibitor, the TGF/activin/nodal signalling pathway inhibitor, and the Wnt signalling pathway activator are added in the culture medium starting from DO until D3 or D4,
the sonic Hedgehog signalling pathway agonist is added to the culture medium starting from D3 to D7 and until D9 or D10, and
optionally a notch pathway inhibitor is added between D9 and D14.

19. The method of claim 14, wherein caudal thoracic and lumbar motor neurons are obtained by exposing the axial progenitors to the RA, the FGFR agonist and the TGF/activin/nodal signalling pathway activator.

20. The method of claim 14, wherein the FGFR agonist is added to the culture medium at a concentration of at least 15 ng/ml for a period of time of at least 24 hours and the TGF/activin/nodal signalling pathway activator is added to the culture medium at a concentration of at least 20 ng/ml for a period of time of at least 24 hours.

21. The method of claim 14 wherein the RA is added to the culture medium for a period of 2 to 11 days.

22. The method of claim 14, wherein:
brachial motor neurons are obtained by addition to the culture medium of the RA alone between D4 and D9 or by addition to the culture medium of RA between D3 and D9 and the FGFR agonist or the TGF/activin/nodal signalling pathway activator between D3 and D4, respectively, at a concentration of at least 100 ng/ml or at least 20 ng/ml, respectively;
anterior thoracic motor neurons are obtained by addition to the culture medium of the RA alone starting from D5 or until D9, or by addition to the culture medium of the RA between D3 and D9 and the FGFR agonist at a concentration of at least 60 ng or the TGF/activin/nodal signalling pathway activator at a concentration of at least 20 ng/ml, respectively, between 24 to 48 hours before addition of the RA to the culture medium,
caudal thoracic motor neurons are obtained by addition to the culture medium of the RA between D3 and D9 and the FGFR agonist at a concentration of at least 100 ng/ml and the TGF/activin/nodal signalling pathway activator at a concentration of at least 20 ng/ml between D3 and D4 of; and
lumbar motor neurons are obtained by addition to the culture medium of the RA between D5 and D9 and the FGFR agonist at a concentration of at least 100 ng/ml and the TGF/activin/nodal signalling pathway activator at a concentration of at least 20 ng/ml, respectively, between D3 or D4 and D5.

23. The method of claim 14, wherein the FGFR agonist is FGFR2.

24. The method of claim 14, wherein the sonic Hedgehog signalling pathway agonist is Smoothened Agonist (SAG).

25. The method of claim 24 wherein the sonic Hedgehog signalling pathway agonist is SAG at a concentration of at least 300 nM.

26. The method of claim 16, wherein the notch pathway inhibitor is DAPT.

27. The method of claim 26 wherein the notch pathway inhibitor is DAPT at a concentration of at least 5 μM.

28. The method of claim 15, wherein the Wnt signalling pathway activator is a Chir-99021 compound or a Wnt3a protein.

29. The method of claim 14, wherein the TGF/activin/nodal signalling pathway activator is GDF11 or GDF8.
